(19) United States
(12) Patent Application Publication
(10) Pub. No.: US 2003/0199494 A1
(43) Pub. Date: Oct. 23, 2003

(54) 1,2,3,5-tetrahydrobenzo'c!azepin-4-one derivatives having muscarinic antagonist activity

(76) Inventors: Paul Evans, Liverpool (GB); Eric James Thomas, Stockport (GB); Robin Havard Davies, Cardiff (GB)

Correspondence Address: WELSH & KATZ, LTD, 120 S RIVERSIDE PLAZA, 22ND FLOOR, CHICAGO, IL, 60606, US

(21) Appl. No.: 10/311,156

(22) PCT Filed: Jun. 14, 2001
(86) PCT No.: PCT/GB01/02594

(30) Foreign Application Priority Data
Jun. 14, 2000 (GB) 0014561.5
Jan. 9, 2001 (GB) 0100600.6

(51) Int. Cl. A61K 31/55; C07D 417/02; C07D 413/02; C 07D 4 3/02; C07D 223/16

(52) U.S. Cl. 514/217.01; 540/593

(57) ABSTRACT

There is disclosed a compound having the formula or a pharmaceutically acceptable salt thereof, wherein: R₁ₐ, R_{1b} and R_{1c} are independently fluorine or hydrogen; R₂ is C₁ to C₁₂ alkyl being straight or branched chain, saturated or unsaturated, mono-substituted or unsubstituted, said substituents being selected from piperidine, pyrroliding, morpholine, thiomorpholine and cycloalkyl of 3 to 7 carbon atoms; a cycloalkyl of 3 to 9 carbon atoms; a cycloalkyl of 3 to 9 carbon atoms having a C₁ to C₆ alkyl substituent; a polycycloalkyl of 2 to 3 rings having 7 to 12 carbons; and phenyl or phenyl substituted with halogen, hydroxy, C₁ to C₆ alkoxy, C₁ to C₆ alkyl, nitro, methylene dioxy or trifluoromethyl; and R₃ is a moiety selected from: (I), (II) or a pyrrolidin-3-yl moiety of the formula (III). The compounds are disclosed for use as muscarinic antagonists with M₃ selectivity.

This invention relates to muscarinic antagonists with M₃ selectivity.

Muscarinic M₃ receptors are located predominantly on smooth muscle and salivary glands, and agents selective for this sub-class of receptors may have therapeutic utility in the treatment of incontinence, disorders of gastro-intestinal motility and as bronchodilators in respiratory disease.

EP-A-0486734 discloses 1-substituted-1-hydroxy-1-aryl-3-(4-substituted-1-piperizinyl)-2-propanones having antimuscarinic activity.

According to the present invention, there is provided a compound having the formula:

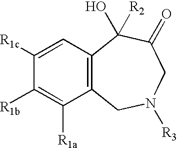

or a pharmaceutically acceptable-salt thereof, wherein:

R₁ₐ, R_{1b} and R_{1c} are independently fluorine or hydrogen;

R₂ is C₁ to C₁₂ alkyl, said alkyl being straight or branched chain, saturated or unsaturated, mono-substituted or unsubstituted, said substituents being selected from piperidine, pyrrolidine, morpholine, thiomorpholine and cycloalkyl of 3 to 7 carbon atoms; a cycloalkyl of 3 to 9 carbon atoms; a cycloalkyl of 3 to 9 carbon atoms (preferably 4 to 9 carbon atoms) having a C₁ to C₆ alkyl substituent; a polycycloalkyl of 2 to 3 rings having 7 to 12 carbons, preferably 7-9 carbon atoms; and phenyl or phenyl singly or multiply substituted (preferably singly or doubly) with halogen, hydroxy, C₁ to C₆ alkoxy, C₁ to C₆ alkyl, nitro, methylene dioxy or trifluoromethyl; and

R₃ is a moiety selected from:

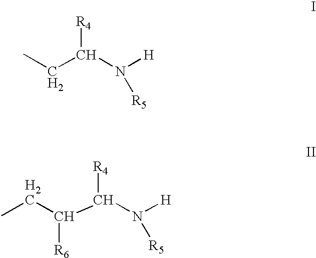

or a pyrrolidin-3-yl moiety of the formula

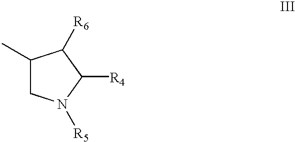

where R₆ is hydroxy or hydrogen;

where one of R₄ and R₅ is hydrogen or lower C1-3 alkyl and the other is selected from:

(a) hydrogen,

(b) phenyl,

(c) phenyl singly or multiply substituted with halogen, hydroxy, C₁ to C₆ alkoxy, C₁ to C₆ alkyl, nitro, methylene dioxy or trifluoromethyl; and

(d) C₁ to C₆ alkyl which may be branched chain or straight, saturated, unsaturated, or cyclic and may be optionally substituted with hydroxy, thienyl, pyrrolyl, pyridyl, furanyl, lower alkoxy or acetoxyalkyl wherein the alkyl group has 1 to 3 carbons, phenyl, phenyl singly or multiply substituted (preferably singly or doubly) with halogen, hydroxy, C₁ to C₆ alkoxy, C₁ to C₆ alkyl, nitro, methylene dioxy or trifluoromethyl.

In an embodiment of the invention, R₂ is not a phenyl or substituted phenyl, R₃ has the structural formula II or III, and one of R₄ and R₅ is hydrogen whilst the other is selected from substituents (a), (b), (c) or (d).

Radical R1a, b, c

In embodiments of the invention, R₁ₐ, R_{1b} and R_{1c} are each fluorine or each hydrogen. In other embodiments, R₁ₐ is hydrogen and either one of R_{1b} and R_{1c} is fluorine and the other is hydrogen or both R_{1b} and R_{1c} are fluorine.

Radical R2

When R₂ is substituted C₁-C₁₂ alkyl, the substituent on the alkyl may additionally be selected from tetrahydrofuran, thiophen and furan. Further, when R₂ is C₁-C₁₂ alkyl, it is preferred that the alkyl is saturated.

In preferred embodiments of the invention, R₂ may be cycloalkyl of 3 to 6 carbon atoms, for example cyclohexyl or cyclobutyl, preferably cyclobutyl. In other preferred embodiments of the invention, R₂ may be phenyl.

Radicals R4 and R5

In addition to the above definition, under alternative (d) for R₄/R₅, the or each alkyl substituent on the phenyl radical may be a C₁-C₁₀alkyl, preferably a C₅-C₈ alkyl, and the or each alkoxy substituent on the phenyl radical may be C₁-C₁₀ alkoxy.

Further, in addition to the above definition under alternative (d) for R₄/R₅, the methylene dioxy substituent may itself be mono or di-substituted by an alkyl having 1 to 10 carbons, preferably dialkyl-substituted where each alkyl has from 1 to 5 carbons.

It is preferred that R₄ is hydrogen and R₅ is selected from amongst the groups (a)-(d) above.

In one embodiment, one of R₄ and R₅ is hydrogen (or methyl in the case of R₅) and the other is selected from hydrogen, C₁ to C₆ alkyl which may be branched chain or straight, saturated, unsaturated, or cyclic and may be optionally substituted with hydroxy, thienyl, pyrrolyl, pyridyl, furanyl, phenyl, phenyl singly or multiply substituted (preferably singly or doubly) with halogen, hydroxy, C₁ to C₆ alkoxy, C₃ to C₆ alkyl or nitro. More preferably, one of R₄ and R₅ is hydrogen and the other is C₁ to C₆ alkyl, benzyl, substituted benzyl or cinnamyl; such as benzyl or 4-substituted benzyl; for example benzyl, 4-chlorobenzyl or 4-methylbenzyl.

In another embodiment., it is preferred that R₄ is hydrogen and R₅ is C₁ to C₆ alkyl substituted by phenyl or phenyl which is singly or multiply substituted (preferably singly or doubly) with halogen, hydroxy, C₁ to C₁₀ alkoxy, C₁ to C₁₀ alkyl or nitro. More preferably, R₅ is benzyl, substituted benzyl or cinnamyl. Most preferably, R₅ is substituted benzyl in which the substituent(s) on the benzyl are independently halo, C₁ to C₁₀ alkoxy or C₁ to C₁₀ alkyl. For example, the benzyl may be substituted by one or two alkyls where the total number of carbon atoms in the alkyl substituent(s) is from 6 to 10. In another example, the benzyl may be substituted by an alkyl radical having from 5-9 carbon atoms and a halo, preferably chloro. Where the benzyl is mono-substituted, this is preferably in the 3- or 4-position. Where the benzyl is di-substituted, this is preferably in the 3- and 4-positions.

Radical R6

It is preferred that R₆ is hydrogen.

### PREFERRED EMBODIMENTS OF THE INVENTION

In a first preferred embodiment of the invention, R₁ₐ, R_{1b} and R_{1c} are independently hydrogen or fluorine, R₂ is cycloalkyl of 3 to 6 carbon atoms or phenyl, R₃ is

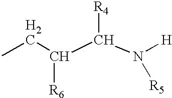

where R₄ is hydrogen and R₅ is selected from C₁ to C₆ alkyl, benzyl, substituted benzyl or cinnamyl, and R₆ is hydrogen or hydroxy. Preferably R₆ is hydrogen.

In this first embodiment, R₂ is preferably cyclohexyl, cyclobutyl or phenyl, more preferably cyclobutyl, and R₅ is preferably C₁ to C₆ alkyl, benzyl, substituted benzyl or cinnamyl, such as methyl, benzyl or 4-substituted benzyl, for example benzyl, 4-chlorobenzyl or 4-methylbenzyl.

Alternatively in this first embodiment (and presently preferred), R₅ is substituted benzyl in which the substituent(s) on the benzyl are independently halo, C₁ to C₁₀ alkoxy or C₁ to C₁₀alkyl. For example, the benzyl may be substituted by one or two alkyls where the total number of carbon atoms in the alkyl substituent(s) is from 6 to 10. In another example, the benzyl may be substituted by an alkyl radical having from 5-9 carbon atoms and a halo, preferably chloro. Where the benzyl is mono-substituted, this is preferably in the 3- or 4-position. Where the benzyl is di-substituted, this is preferably in the 3- and 4-positions.

In a second embodiment (which is presently less preferred than the first embodiment) R₁ₐ, R_{1b} and R_{1c} are independently hydrogen or fluorine, R₂ is cycloalkyl of 3 to 6 carbon atoms or phenyl, R₃ is

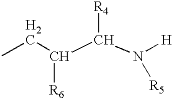

where R₅ is hydrogen or methyl and R₄ is C₁ to C₆ alkyl, benzyl, substituted benzyl or cinnamyl, and R₆ is hydroxy or hydrogen, preferably hydrogen. In this second embodiment, R₂ is preferably cyclohexyl, cyclobutyl or phenyl, more preferably cyclobutyl, and R₄ is preferably C₁ to C₆ alkyl, benzyl, substituted benzyl or cinnamyl, such as methyl, benzyl or 4-substituted benzyl, for example benzyl, 4-chlorobenzyl or 4-methylbenzyl.

In a third embodiment R₁ₐ, R_{1b} and R_{1c} are independently hydrogen or fluorine, R₂ is cycloalkyl of 3 to 6 carbon atoms or phenyl, R₃ is

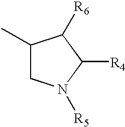

where R₄ is hydrogen and R₅ is selected from C₁ to C₆ alkyl, benzyl, substituted benzyl or cinnamyl, and R₆ is hydroxy or hydrogen.

In this third embodiment, R₂ is preferably cyclohexyl, cyclobutyl or phenyl, more preferably cyclobutyl, and R₅ is preferably C₁ to C₆ alkyl, benzyl, substituted benzyl or cinnamyl, such as methyl, benzyl or 4-substituted benzyl, for example benzyl, 4-chlorobenzyl or 4-methylbenzyl.

Alternatively in this third embodiment (and presently preferred), R₅ is substituted benzyl in which the substituent(s) on the benzyl are independently halo, C₁ to C₁₀ alkoxy or C₁ to C₁₀ alkyl. For example, the benzyl may be substituted by one or two alkyls where the total number of carbon atoms in the alkyl substituent(s) is from 6 to 10. In another example, the benzyl may be substituted by an alkyl radical having from 5-9 carbon atoms and a halo, preferably chloro. Where the benzyl is mono-substituted, this is preferably in the 3- or 4-position. Where the benzyl is di-substituted, this is preferably in the 3- and 4-positions.

In a fourth embodiment (which is presently less preferred than the third embodiment) R₁ₐ, R_{1b} and R_{1c} are independently hydrogen or fluorine, R₂ is cycloalkyl of 3 to 6 carbon atoms or phenyl, R₃ is a pyrrolidin-3-yl moiety having the following structure:

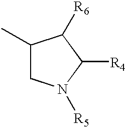

where R₅ is hydrogen or methyl and R₄ is C₁ to C₆ alkyl, benzyl, substituted benzyl or cinnamyl, and R₆ is hydroxy. In this fourth embodiment, R₂ is preferably cyclohexyl, cyclobutyl or phenyl, more preferably cyclobutyl, and R₄ is preferably C₁ to C₆ alkyl, benzyl, substituted benzyl or cinnamyl, such as methyl, benzyl or 4-substituted benzyl, for example benzyl, 4-chlorobenzyl or 4-methylbenzyl.

In a fifth embodiment R₁ₐ, R_{1b} and R_{1c} are independently hydrogen or fluorine, R₂ is cycloalkyl of 3 to 6 carbon atoms or phenyl, R₃ is a moiety having the following structure:

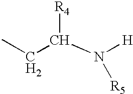

where R₄ is hydrogen and R₅ is selected from C₁ to C₆ alkyl, benzyl, substituted benzyl or cinnamyl.

In this fifth embodiment, R₂ is preferably cyclohexyl, cyclobutyl or phenyl, more preferably cyclobutyl, and R₅ is preferably C₁ to C₆ alkyl, benzyl, substituted benzyl or cinnamyl, such as methyl, benzyl or 4-substituted benzyl, for example benzyl, 4-chlorobenzyl or 4-methylbenzyl.

Alternatively in this fifth embodiment (and presently preferred), R₅ is substituted benzyl in which the substituent(s) on the benzyl are independently halo, C₁ to C₁₀alkoxy or C₁ to C₁₀alkyl. For example, the benzyl may be substituted by one or two alkyls where the total number of carbon atoms in the alkyl substituent(s) is from 6 to 10. In another example, the benzyl may be substituted by an alkyl radical having from 5-9 carbon atoms and a halo, preferably chloro. Where the benzyl is mono-substituted, this is preferably in the 3- or 4-position. Where the benzyl is di-substituted, this is preferably in the 3- and 4-positions.

In a sixth embodiment (which is presently less preferred than the fifth embodiment) R₁ₐ, R_{1b} and R_{1c} are independently hydrogen or fluorine, R₂ is cycloalkyl of 3 to 6 carbon atoms or phenyl, R₃ is

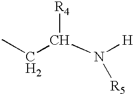

where R₅ is hydrogen or methyl and R₄ is C₁ to C₆ alkyl, benzyl, substituted benzyl or cinnamyl. In this sixth embodiment, R₂ is preferably cyclohexyl, cyclobutyl or phenyl, more preferably cyclobutyl, and R₄ is preferably C₁ to C₆ alkyl, benzyl, substituted benzyl or cinnamyl, such as methyl, benzyl or 4-substituted benzyl, for example benzyl, 4-chlorobenzyl or 4-methylbenzyl.

In each of the first to sixth embodiments described above, it is preferred that R₁ₐ is hydrogen and either one of R_{1b} and R_{1c} is fluorine and the other is hydrogen or both R_{1b} and R_{1c} are fluorine.

As used herein, unless otherwise specified, lower alkyl and lower alkoxy refer to groups having 1 to 6 carbons. The invention also relates to the pharmaceutically acceptable salts of the foregoing compounds and to pharmaceutical compositions containing effective amounts of such compounds; the compounds and compositions may be used for the manufacture of a medicament for the treatment of bladder disorders.

The compounds of the invention may be used in the neutral form. Alternatively, the compounds may be used in the form of pharmaceutically acceptable salts. Salts of the compounds of the invention include the acid salts such as the hydrochloride, sulfate, phosphate, nitrate, methanesulfonate and tartrate salts. Other pharmaceutically acceptable salts are also included in the invention, as are the various possible hydrates of each of the compounds. As will be understood by those skilled in the art, compounds of this invention may be present as d or l optical isomers as well as racemic mixtures thereof. Further, some of the compounds in which R₁ is a substituted cycloalkyl or a polycycloalkyl may be present as diastereoisomers which may be resolved into optical isomers. Resolutions of optical isomers may be accomplished by fractional crystallization of their salts with optically active acids such as, for example, tartaric, camphor-10-sulfonic, O,O-dibenzoyltartaric, O,O-di(p-toluoyl) tartaric, menthyloxyacetic, camphoric, or 2-pyrrolidone-5-carboxylic acids of N-acetyltryptophane from appropriate solvents. They may also be prepared by stereoselective synthesis or by chromatographic techniques using chiral substrates or derivatives. Unless otherwise specified in the claims, it is intended to include all isomers, whether separated or mixtures thereof.

Preferred isomers have the following stereochemistry:

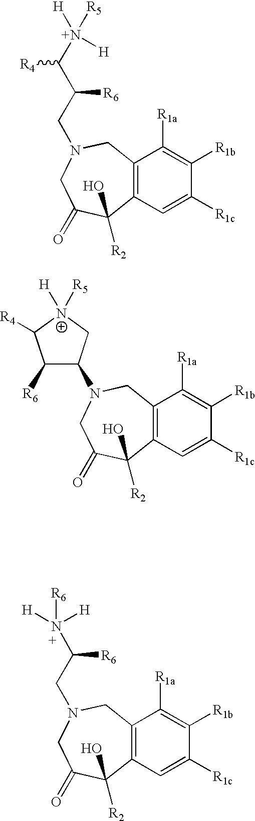

The protonated form of the respective R₃ side chains is shown.

The compounds of the invention may be administered in a variety of pharmaceutical preparations well known to those skilled in the pharmaceutical art. For parenteral administration, the compounds may be prepared in aqueous injection solutions which may contain antioxidants, buffers, bacteriostats, and other additives commonly employed in such solutions. Extemporaneous injection solutions may be prepared from sterile pills, granules or tablets which may contain diluents, dispersing and surface active agents, binders, and lubricants as well as the compound of the invention.

In the case of oral administration, fine powders or granules of the compound of the invention may be formulated with diluents and dispersing and surface active agents, and may be prepared in water, a syrup, capsules, cachets, a non-aqueous suspension or an emulsion. In dry forms, optional binders and lubricants may be present. The compositions may also include flavorants, preservatives, suspending, thickening and emulsifying agents and other pharmaceutically acceptable additives. Granules or tablets for oral administration may be coated. In general, the compositions of the invention include the compounds of the invention in a pharmaceutically effective amount in a pharmaceutically acceptable carrier.

The compounds are useful as antimuscarinic agents selective for the muscarinic M₃ receptor; more particularly, they are useful as bronchodilators, as antispasmodics, antisecretory agents, have antiulcer activity and are useful in the treatment of patients suffering from neurogenic bladder disorders. The compounds are administered in pharmaceutically effective amounts. Daily dosages will generally be at a rate of 5 to 100 mg/day, more specifically 10 to 40 mg/day. Because of their duration of action the compounds may be administered less frequently than certain prior art antimuscarinic agents, particularly those used in the treatment of neurogenic bladder disorder.

The compounds of the invention may be tested to determine their muscarinic activity in accordance with the procedure set forth in EP-A-0486734. The compounds may also be tested for their M₁, M₂ and M₃ receptor activity using the assays set forth after the examples below.

According to a process aspect of the present invention, the compounds of the invention process may be synthesised by a process which includes the step of subjecting a compound of the formula (X):

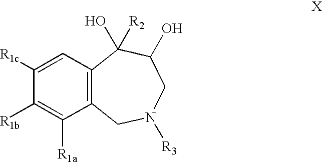

in which R1a, R1b, R1c and R2 are as defined above and R3 is as defined above suitably protected, to oxidation conditions sufficient to oxidise the alcohol group at the 4-position of the benzo[c]azepine core to a ketone group.

For example, the R₃ groups may be protected as follows

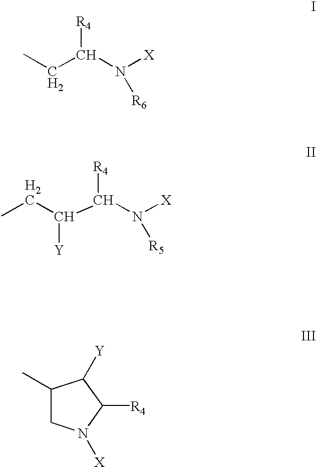

in which Y is hydrogen or a hydroxy protecting group such as acetyl, and X is an amine protecting group such as a trifluoroacetamide or a nosyl group. In Formula III the nitrogen group only requires protection where R₅ in the final molecule is hydrogen.

The oxidation step to oxidise the alcohol group at the 4-position of the benzo[c]azepine core to a ketone group is preferably a Swern oxidation step (K. Takahashi, M. Ogata, J. Org. Chem, 1987, 52, 1877).

In this process aspect of the invention, the compound X may be made by a process in which a compound of the formula XI

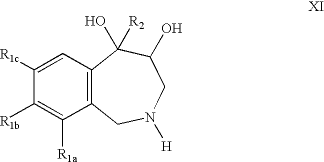

is subjected to a reductive amination with an aldehyde/ketone corresponding to R₃, suitably protected. For example, one of the following protected aldehyde or ketone may be employed:

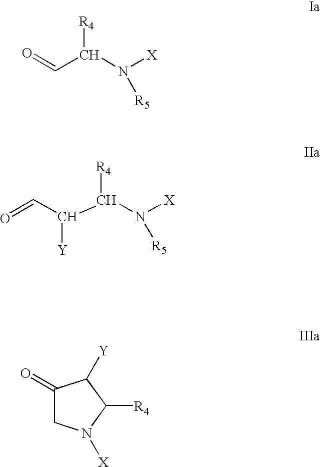

where X and Y are as defined above.

This reductive amination may be accomplished following the procedure of Borch et al. (R. F. Borch, M. D. Bernstein, H. D. Hurst, J. Am. Chem. Soc., 1971, 93, 2897) using the reagent NaBH₃CN at an optimum pH of about 6.

Details of the routes to the compound XI and reagents Ia, IIa and IIIa are discussed in detail below.

The following section concerns the synthesis of compounds in accordance with the invention in which each of R₁ₐ, R_{1b} and R_{1c} is hydrogen, and uses as starting material the commercially available compound phthalide (isobenzofuran).

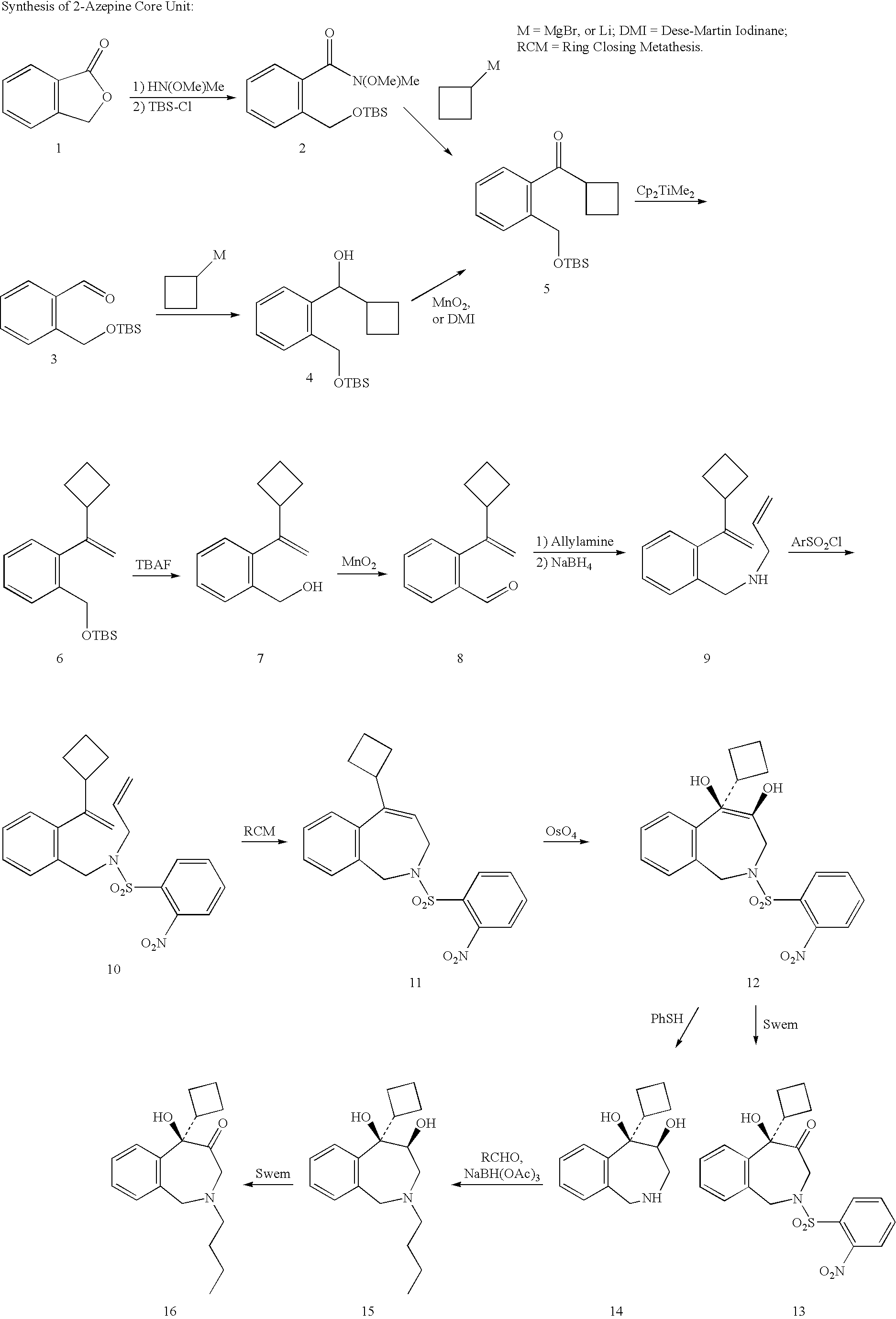

Ketone 5 is accessed in two ways via the Weinreb amide 2 or via an addition-oxidation protocol. 6 is prepared by treatment of 5 with the Petasis reagent (Cp₂TiMe₂). Deprotection, oxidation followed by a two step reductive amination gives the dialkenyl amine 9. Nitrogen derivatisation with 2-nitrophenyl sulfonyl chloride gives 10 which is converted to the dihydroazepine 11 using tricyclohexylphosphine-[1,3-bis(2,4,6-trimethylphenyl)-4,5-dihydroimidazol-2-ylidene][benzylidene]ruthenium dichloride. This ring closing step follows the methodology developed by Grubbs using catalysts based on ruthenium (P. Schwab, R. H. Grubbs, J. W. Ziller, J. Am. Chem. Soc., 1996, 118, 100; S.T. Nguyen, R. H. Grubbs, J. W. Ziller, J. Am. Chem. Soc., 1993, 115, 9858; E. L. Dias, S. T. Nguyen, R. H. Grubbs, J. Am. Chem. Soc., 1998, 63, 824). Other metal catalysts for the construction of cyclic amines are known in the art. Dihydroxylation using OsO₄ gives the diol 12. The sulfonyl group in 12 is removed using thiophenol giving the amino diol 14. In the first scheme the reductive coupling of butyraldehyde to give 15 is shown, although outside the scope of the invention. The coupling of different side chains is described in more detail below. Swern oxidation generates the β-amino ketone 16.

The aldehydes and ketones containing the second amino group were synthesised as follows:

C-3 Side Chain 20:

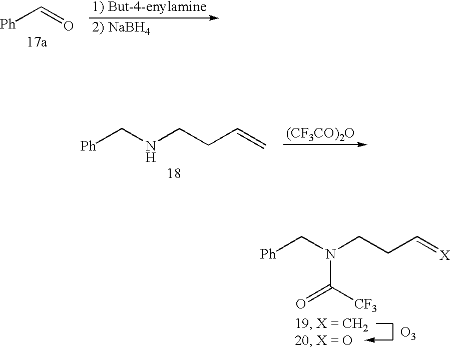

Two step reductive amination between benzaldehyde 17a and but-4-enylamine gives the amine 18. Protection of 18 as the trifluoroacetamide gives the alkene 19, treatment with ozone gives the aldehyde 20 on reductive work-up.

The hydroxy C3 side chain may be synthesised as indicated below with an asymmetric hydroxylation providing enantiomerically enriched material. The key steps are the reductive amination of the aryl aldehyde with allylamine 201 to give the secondary amine 202. Protection of this either as its o-nitrobenzenesulfonyl or trifluoroacetyl derivative 203 followed by asymmetric hydroxylation to provide the diol 204. Regioselective O-silylation and then acetylation of the secondary alcohol will provide the acetate 205 which on desilylation and oxidation will give the required aldehyde 206. Reductive coupling of the aldehyde then proceeds as described above.

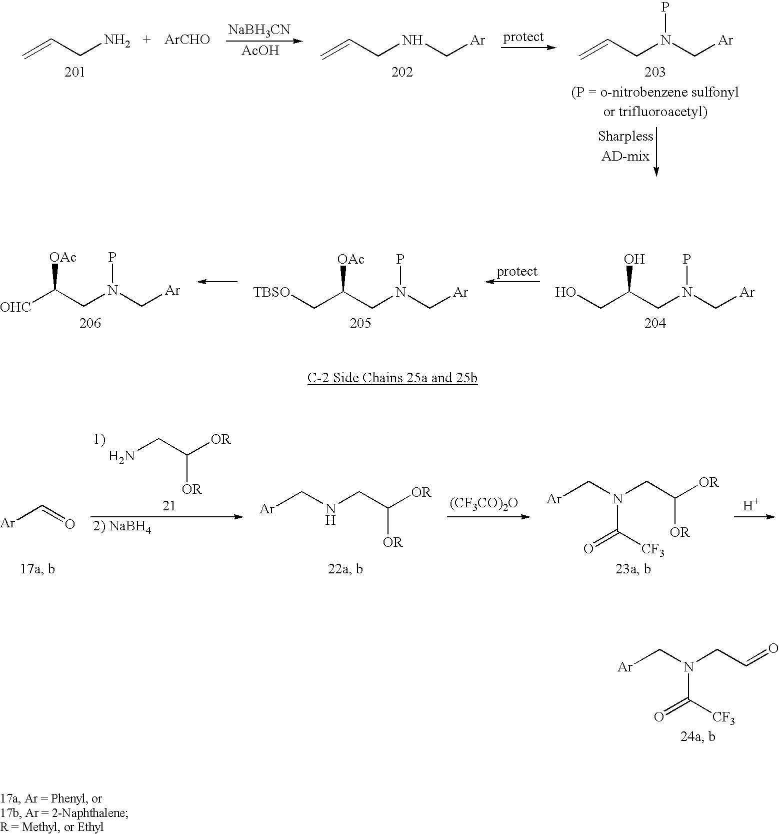

Following a similar sequence described above reductive amination of benzaldehyde 17a and 2-naphthylaldehyde 17b with the acetal 21 gives the amines 22a and 22b. Protection of the amino group with trifluoroacetic anhydride gives the amides 23a and 22b. Acetal deprotection under acidic catalysis furnishes the aldehydes 24a and 24b.

Pyrrolidine Side Chains 31 and 33:

Reference is made to the following reaction scheme. Trans-4-Hydroxyproline 25 is protected with the tert-butyloxy carbonyl group (Boc) 26 before acid activation as the Weinreb amide and hydroxyl protection as the tert-butyldimethylsilyl ether (TBS) giving the known amide 27. Organometallic addition to 27 with phenyl magnesium bromide (PhMgBr) gives the ketone 28.

Reduction of the amide 27 with diisobutylaluminium hydride gives the aldehyde 29. Carbonyl reduction of 28 and 29 generates the protected pyrrolidines 30 and 31. TBS removal followed by Swern oxidation generates the Boc protected ketones 31 and 33.

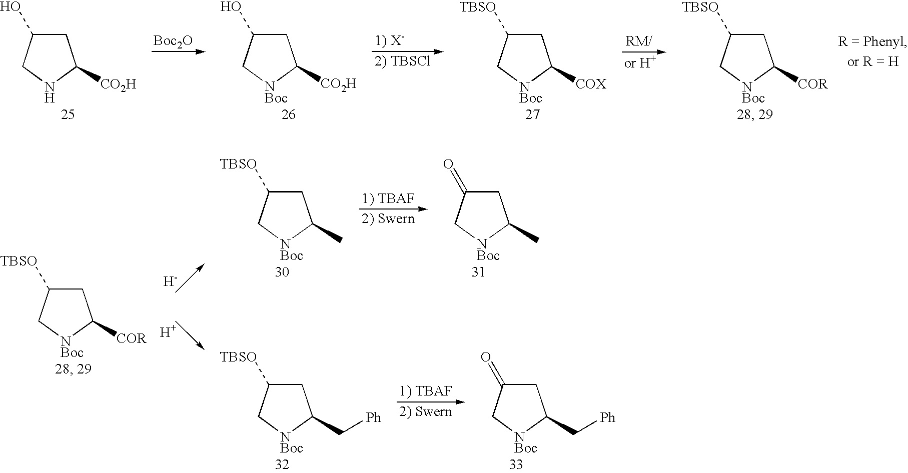

An alternative synthesis of the pyrrolidine side chains is as follows.

With reference to the following reaction scheme, the starting material hydroxyproline 101 is bis-protected and converted into the ester 102. Reduction-oxidation to the aldehyde 103 followed by addition of an aryl Grignard reagent will give the alcohol 104, as a mixture of diastereoisomers, which is reduced using Barton chemistry (e.g. conversion into the thionocarbonate followed by reduction using tributyltin hydride) to give the pyrrolidine derivative 105. At this stage the t-Boc group will be replaced by a trifluoroacetate and desilylation and oxidation will give the ketone 106. Reductive coupling of the ketone then proceeds as described above.

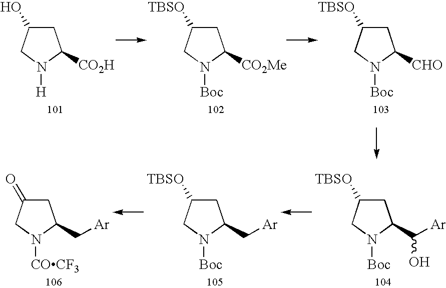

Side Chain Coupling to Azepinyl Nucleus 14:

The side chains whose synthesis is described above are then coupled to the functionalised azepinyl nucleus 14 under one-pot reductive amination conditions. This furnishes the diols 34a-c and 36a,b. Swern oxidation of the secondary hydroxyl group and deprotection of the trifluoroacetamide 34a-c, or tert-butyloxycarbonyl 35a,b gives the azepines 35a-c and 37a,b.

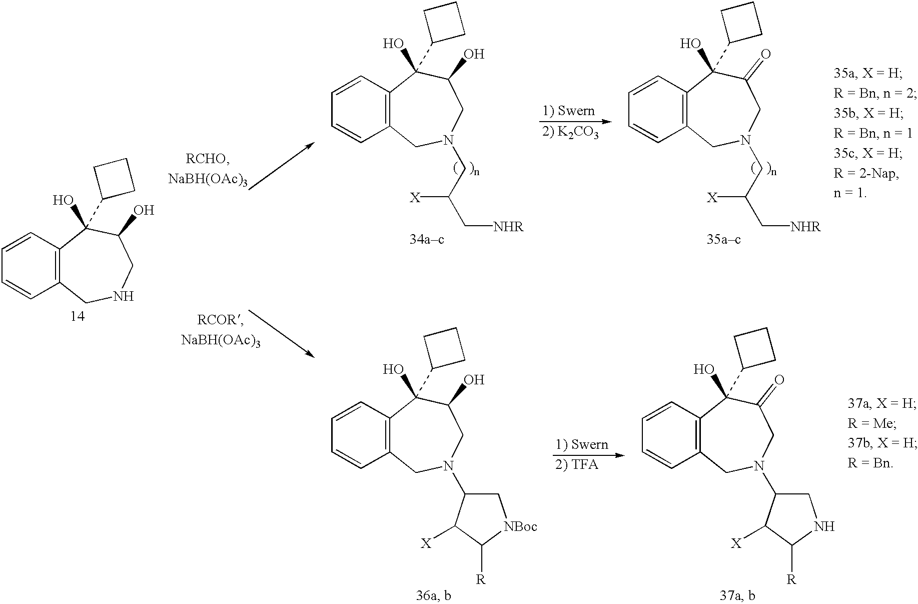

The 5-fluoro analogues may be synthesised as follows:

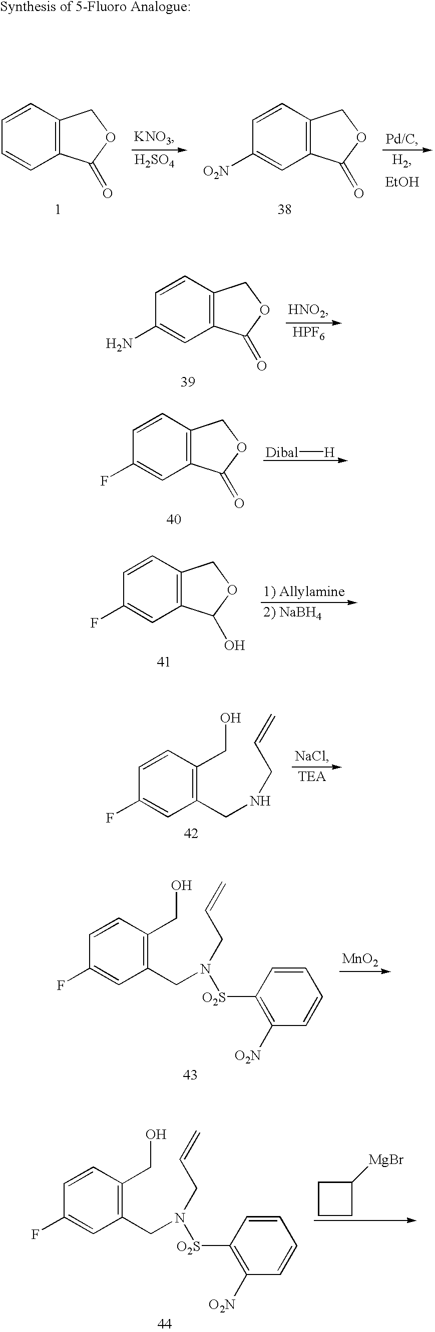

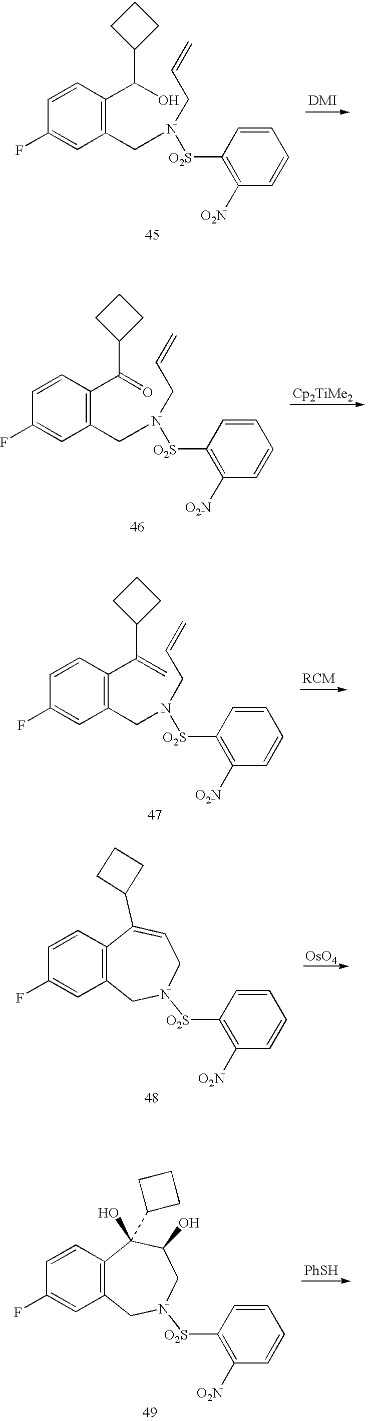

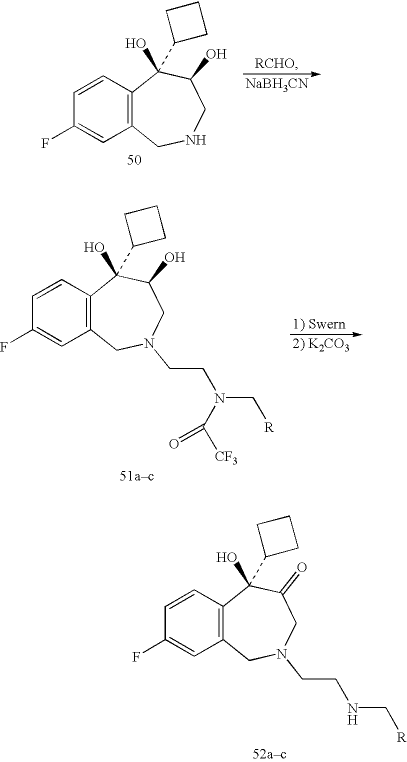

Phthalide 1 is regio-selectively nitrated to give 38. Reduction of the nitro group gives the aniline 39, which is converted into the known 5-fluorophthalide 40. Diiisobutylaluminium hydride reduction of the lactone in 40 gives the lactol 41. Reductive amination of 41 with allylamine gives the amine 42, which is chemo-selectively converted to its 2-nitrophenylsulfonyl derivative 43. Manganese dioxide oxidation of 43 gives the aldehyde 44 which reacts with cyclobutylmagnesium bromide to give the benzylic alcohol 45. Oxidation giving the ketone 46 followed by methylenation with Petasis's reagent gives the dialkenyl cyclisation precursor 47. Ring closing olefin metathesis with the imidazoyl based ruthenium benzylidene catalyst gives the dihydroazepine 48. Dihydroxylation with OsO₄ gives the diol 49, which is converted into the amine 50. 50 is then coupled with the acyclic side chains 20 and 24a,b under the one-pot reductive amination conditions to give the diols 51a-c. Oxidation, followed by trifluoroacetamide deprotection gives the 4-fluoro bis-amines 52a-c.

An alternative (less preferred) synthetic pathway to that described above is as follows. These pathways may be generalized by the skilled person where necessary.

For compounds where R₃ is as follows:

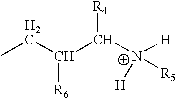

the following synthetic pathway may be employed.

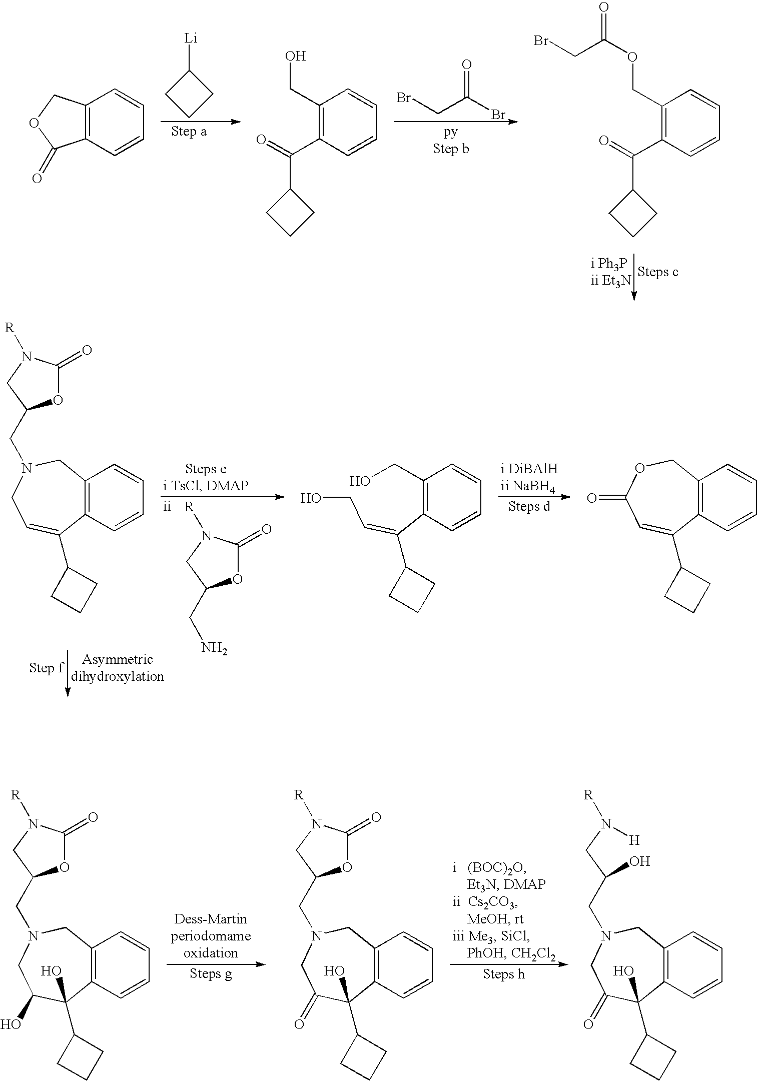

Step a Reaction of the cyclobutyl lithium (or other organometallics) with the known lactone is conducted at −78° C., with slow inverse addition of the reagent.

Step b Reaction of the ketoalcohol with bromacetyl bromide and pyridine (mole ratio 1:1:1), gives an unstable bromoester; which is utilised immediately.

Steps c The bromoester is dissolved in acetonitrile at room temperature and treated with triphenylphosphine. After 3 days stirring at room temperature triethylamine is added and after a further week the ε-lactone is isolated.

Steps d The lactone may be reduced in one step, but the better yields are achieved by use of a two step protocol via the lactol.

Steps e The bis(tosylate) is prepared at −20° C. warming to room temperature to minimise formation of a cyclic ether. Formation of the seven membered ring is performed under high dilution conditions (0.1 mmolar) in DMSO to minimise dimer formation. The methodology is illustrated by the oxazolidinone protected amino alcohol, but other protecting groups may be employed such as carbonenzyloxy.

Steps f The diol moiety is installed by use of Sharpless asymmetric dihydroxylation methodology using the AD-mix α or a comparable reagent.

Steps g Oxidation of the diol is performed under mild conditions using the Dess-Martin periodinane reagent.

Steps h The oxazolidinone ring is cleaved directly under acidic or basic conditions. In the case where R=H, the three step method indicated in the scheme is the preferred method.

For compounds in which R₃ is as follows:

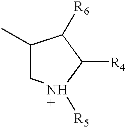

the same pathway as given above in the immediately preceding section may be used except that a different side chain is used in the N-alkylation step (step (e)).

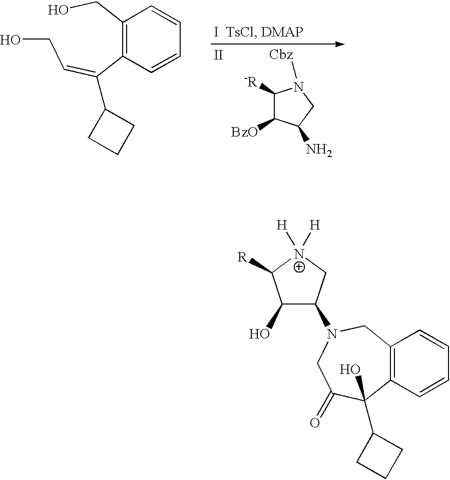

The invention will now be illustrated by the following examples.

In the examples, flash column chromatography was performed using Merck silica gel (60H; 40-60μ, 230-240 mesh). Thin layer chromatography (TLC) was carried out using glass backed plates coated with Merck HF_{254/366} silica gel. The spots were visualised using ultraviolet radiation, treatment with basic permanganate solution, or acidic ethanolic anisaldehyde solution. Petroleum ether (Pet) was redistilled before use and refers to the fraction boiling between 40 and 60° C. Tetrahydrofuran was dried over sodium-benzophenone and was distilled prior to use. Dichloromethane was dried over CaH₂ and was distilled before use.

Mass spectra, either electron impact (EI), or chemical ionisation using ammonia (CI), were recorded by Val Boote using a Fisons VG Trio 200 spectrometer. High resolution mass spectra were recorded by Peter Kobryn on a Kratos Concept IS spectrometer. Microanalyses were performed using a Carlo-Erba combustion analyser for C, H and N. Infra-red spectra were recorded on a Genesis FTIR spectrometer on NaBr plates, either neat, or as evaporated films. Proton, proton-decoupled carbon and fluorine nuclear magnetic resonance spectra were recorded on either a Varian (400 MHz), Varian INOVA 300 (300 MHz), or a Varian Gemini 200 (200 MHz) spectrometer. Where applicable proton assignment was facilitated using correlation spectroscopy (COSY). Residual non-deuterated solvent was used as an internal standard and the chemical shifts are quoted in ppm down field from tetramethylsilane. Signal splitting patterns are described as singlets (s), doublets (d), doublet of doublets (dd), doublet of double doublets (ddd), triplets (t), doublet of triplets (dt), quartets (q), or multiplets (m). The coupling constants (o are given in Hertz (Hz).

### EXAMPLE 1

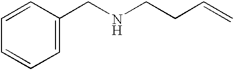

N-Benzyl-N-(3-butenyl) amine 1:

According to literature⁷⁹ at 0° C. AlCl₃ (18.0 g, 0.135 mol, 1 eq.) in dry Et₂O (200 cm³) was treated initially with LAH (5.12 g, 0.135 mol, 1 eq.) and then after 0.5 h allyl cyanide (9.3 g, 0.135 mol, 1 eq.) was added dropwise. Stirring was maintained for 2 h at 0° C. before H₂O (20 cm³) was added followed by 4 M NaOH (20 cm³) and H₂O (60 cm³). The solid residue was filtered, washing with Et₂O (2×50 cm³). The volatile amine was stripped in vacuo with care and added directly to a solution of benzaldehyde (14 cm³, 0.137 mol, 1.01 eq.) in DCM (200 cm³) with MgSO₄ (20 g). Stirring was continued at room temperature for 24 h. Filtration followed by solvent removal gave the imine, which was reduced directly. The imine in MeOH (100 cm³) was treated portionwise with NaBH₄ (5.1 g, 0.134 mol, 1 eq.) and stirring was continued for 2 h. The reaction mixture was concentrated in vacuo and Et₂O (100 cm³) and H₂O (100 cm³) were added. The resultant aqueous phase was further extracted with Et₂O (2×100 cm³) and the combined organic phases were dried over MgSO₄. Filtration and solvent removal under reduced pressure gave 1 (5.9 g, 44%) as a yellow liquid. m/z (CI) 162 (MNH₄⁺, 100%); δ_{H} (300 MHz, CDCl₃) 2.25 (2H, q, J 7.0 Hz, CH₂), 2.76 (2H, t, J 7.0 Hz, CH₂), 3.82 (2H, s, CH₂), 5.02-5.19 (2H, m, CH₂), 5.76-5.94 (1H, m, CH), 7.23-7.44 (5H, m, ArH).

### EXAMPLE 2

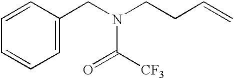

N-Benzyl-N-(3-butenyl)-2,2,2-trifluoroacetamide 2:

At 0° C. a solution of amine 1(5.9 g, 36.65 mmol, 1 eq.) and TEA (25.0 cm³, 179.37 mmol, 5 eq.) in DCM (100 cm³) was treated with a solution of (CF₃CO)20 (7.8 cm³, 55.22 mmol, 1.5 eq.) added via a dropping funnel. The mixture was stirred for 3 h at 0° C. to room temperature. HO (100 cm) was added and the resultant aqueous phase was further extracted with DCM (2×100 cm³). The combined organic extracts were dried over MgSO₄. Filtration, solvent removal under reduced pressure and purification by flash column chromatography (Pet:Et₂O; 9:1) gave 2 (6.42 g, 68%) as a yellow liquid. R_{f}=0.3 (Pet:Et₂O; 9:1); m/z (CI) 275 (MNH₄⁺, 100%), 258 (MH⁺, 40%); found (EI) 257.1022, C₁₃H₁₄NOF₃ requires 257.1027 (−1.9 ppm); δ_{H} (300 MHz, CDCl₃) 2.23-2.42 (2H, m, CH₂), 3.34-3.45 (2H, m, CH₂), 4.62 (2H, s, CH₂), 4.71 (2H, s, CH₂), 5.05-5.18 (2H, m, CH₂), 5.63-5.81 (1H, m, CH), 7.11-7.22 (5H, m, ArH); ¹H-NMR spectrum complicated due to restricted rotation.

### EXAMPLE 3

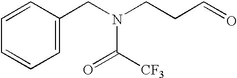

N-Benzyl-N-(3-oxopropyl)-2,2,2-trifluoroacetamide 3:

At −78° C. a solution of 2 (2.01 g, 7.82 mmol, 1 eq.) in DCM (20 cm³) was treated with a steady stream of ozone gas for 0.5 h. At this point TLC analysis indicated consumption of 2. The excess ozone was purged under a flow of oxygen and DMS (3 cm, 40.86 mmol, 5.2 eq.) was added. The reaction mixture was warmed to room temperature and stirred for 15 h. Solvent removal in vacuo and flash column chromatography (Pet:EtOAc; 4:1, 1% TEA) gave 3 (1.72 g, 85%) as a clear liquid. R_{f}=0.2 (Pet:EtOAc; 4:1); ); υₘₐₓ (neat/cm⁻¹) 3066, 3034, 2944, 2836, 2733, 1724, 1690, 1453, 1377, 1204, 1147; m/z (CI) 277 (MNH₄⁺, 100%), 260 (MH⁺, 100%); found 277.1167, C₁₂H₁₂NO₂F₃.NH₄ requires 277.1164 (+1.1 ppm); δ_{H} (300 MHz, CDCl₃) 2.75-2.84 (2H, m, CH₂), 3.61 (2H, t, J 6.5 Hz, CH₂), 3.76 (2H, t, J 7.0 Hz, CH₂), 4.66 (2H, s, CH₂), 4.73 (2H, s, CH₂), 7.25-7.47 (5H, m, ArH), 9.74 (1H, s(br), CHO); ¹H-NMR spectrum complicated due to restricted rotation (60:40).

### EXAMPLE 4

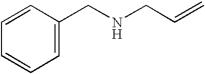

N-Allyl-N-benzylamine 4:

A mixture of benzaldehyde (9.8 cm³, 96.0 mmol, 1 eq.), allylamine (10.8 cm³, 143.9 mmol, 1.5 eq.) and MgSO₄ (20 g) in DCM (100 cm³) were stirred together at room temperature for 15 h. Filtration and solvent removal under reduced pressure gave the imine (ca. 96 mmol) which was dissolved in MeOH (100 cm³). At room temperature NaBH₄ (3.65 g, 96.1 mmol, 1 eq.) was added in portions. Stirring was continued for 2 h before approximately half the solvent was removed under reduced pressure. Et₂O (100 cm³) and H₂O (100 cm³) were added and the mixture was basified with 1 M NaOH (ca. pH 12). The aqueous layer was further extracted with Et₂O (2×100 cm³) and the combined ethereal extracts were dried over MgSO₄. Filtration and solvent removal in vacuo afforded the amine 4 (8.94 g, 64%) as a clear liquid. m/z (CI) 148 (MH⁺, 100%); δ_{H} (200 MHz, CDCl₃) 1.5 (1H, s(br), NH), 3.32 (2H, d, J 6.5 Hz, CH₂), 4.82 (2H, s, CH₂), 5.07-5.32 (2H, m, CH₂), 5.82-6.08 (1H, m, CH), 7.19-7.45 (5H, m, ArH).

### EXAMPLE 5

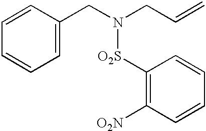

N-Allyl-N-benzyl-2-nitrophenylsulfonamide 5:

At room temperature a mixture of 4 (950 mg, 6.46 mmol, 1 eq.), TEA (1.8 cm^{3, 12.91} mmol, 2 eq.) and DMAP (ca. 2 mg) in DCM (20 cm³) was treated with NsCl (1.43 g, 6.45 mmol, 1 eq.). Stirring was continued for 3 h before H₂O (50 cm³) and Et₂O (50 cm³) were added. The resultant aqueous phase was extracted further with Et₂O (2×50 cm³) and the total organic extracts were dried over MgSO₄. Filtration followed by solvent removal in vacuo afforded the crude sulfonamide which was purified by flash column chromatography (Pet:EtOAc; 5:1) to give 5 (1.63 g, 76%) as a viscous clear oil. R_{f}=0.25 (Pet:EtOAc; 5:1); m/z (CI) 350 (MNH₄⁺, 100%); found 350.1176, C₁₆H₁₆N₂O₄S.NH₄ requires 350.1174 (+0.6 ppm); δ_{H} (300 MHz, CDCl₃) 3.77 (2H, d, J 6.5 Hz, CH₂), 4.45 (2H, s, CH₂), 4.95-5.08 (2H, m, CH₂), 5.50 (1H, ddd app. qt, J 6.5, 10.0, 17.0 Hz, CH), 7.19-7.25 (5H, m, ArH), 7.51-7.65 (3H, m, ArH), 7.93 (1H, d, J 7.5 Hz, ArH); δ_{C} (75 MHz, CDCl₃) 49.2, 50.3, 119.6, 124.2, 127.8, 128.3, 128.6, 130.9, 131.7, 131.8, 133.5, 134.0, 135.3, 147.8.

### EXAMPLE 6

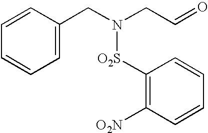

N-Benzyl-N-(2-oxoethyl)-2-nitrophenylsulfonamide 6:

A solution of 5 (1.11 g, 3.34 mmol, 1 eq.) in DCM (25 cm³) at −78° C. was treated with a steady stream of ozone gas until TLC analysis indicated no remaining starting material (ca. 0.5 h). The excess ozone was purged under a flow of oxygen before DMS (4.0 cm³, 54.47 mmol, 16 eq.) was added and the mixture was allowed to warm to room temperature and stirred for 15 h. Evaporation of the solvent under reduced pressure followed by flash column chromatography (Pet:EtOAc; 1:1) gave 6 (1.04 g, 92%) as a colourless solid. For microanalysis 6 was recrystallised from EtOAc and petroleum ether. R_{f}=0.25 (streak) (Pet:EtOAc; 5:1); υₘₐₓ (CDCl₃/cm⁻¹) 3055, 2986, 2831, 1735, 1546, 1371, 1266, 1166; m/z (FAB) 690 (M₂Na⁺, 90%); δ_{H} (300 MHz, CDCl₃) 4.11 (2H, s, CH₂), 4.65 (2H, s, CH₂), 7.25-7.38 (5H, m, ArH), 7.65-7.74 (3H, m, ArH), 8.11 (1H, d, J 7.5 Hz, ArH), 9.39 (1H, s, CHO); δ_{C} (75 MHz, CDCl₃) δ 2.6, 55.5, 124.3, 128.6, 128.7, 128.9, 130.9, 131.9, 133.0, 133.8, 134.1, 147.0, 196.4; found C, 53.6; H, 4.5; N, 8.4; S, 9.6%; C₁₅H₁₄N₂O₅S requires, C, 53.9; H, 4.2; N. 8.4; S, 9.6%.

### EXAMPLE 7

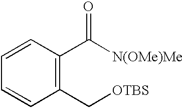

2-(tert-Butyldimethylsilanyloxymethyl)-N-methoxy-N-methylbenzamide 7:

At 0° C. under argon a 2 M solution of AlMe₃ in hexane (32 cm³, 64.5 mmol, 2 eq.) was added dropwise over ca. 0.25 h to a suspension of HCl.NH(OMe)Me (6.29 g, 64.5 mmol, 2 eq.) in DCM (60 cm³). During the addition of 0.5 eq. of AlMe₃ a vigorous gas evolution ensued. The now clear mixture was stirred for 1 h at 0° C. before a solution of phthalide (isobenzofuran) (4.32 g, 32.2 mmol, 1 eq.) in DCM (20 cm³) was added. Stirring was maintained for 7 h during which time room temperature was reached. Saturated sodium potassium tartrate solution (100 cm³) was cautiously added. The resultant aqueous layer, obtained after separation was further extracted with DCM (2×100 cm³). The combined organic extracts were washed with sat. brine solution (100 cm³) and dried over Na₂SO₄. Filtration followed by solvent removal in vacuo gave the crude Weinreb amide which was directly O-protected in order to minimise re-lactonisation. Thus, at room temperature the crude amide (ca. 32.2 mmol, 1 eq.) was dissolved in DCM (50 cm³) and treated with TBDMS-Cl (4.9 g, 32.2 mmol, 1 eq.) and imidazole (4.4 g, 64.5 mmol, 2 eq.). Stirring was continued for 15 h. Water (100 cm³) and DCM (50 cm³) were added and the resultant aqueous layer was further extracted with DCM (100 cm³). The combined organic extracts were dried over MgSO₄ before filtration and solvent removal in vacuo gave the crude product. Purification by flash column chromatography (Pet:EtOAc; 5:1→Pet:EtOAc; 3:1) gave the Weinreb amide 7 (5.93 g, 60%) as a clear liquid. R_{f}=0.3 (Pet:EtOAc; 4:1); υₘₐₓ (neat/cm⁻¹) 3064, 2954, 2893, 2857, 1650, 1463, 1416, 1383, 1257, 1119, 1081; m/z (CI) 310 (MH⁺, 100%); found 310.1835, C₁₆H₂₇NO₃Si.H requires 310.1838 (+0.9 ppm); □_{H} (300 MHz, CDCl₃) 0.00 (6H, s, CH₃), 0.84 (9H, s, CH₃), 3.19 (3H, s, CH₃), 3.44 (3H, s(br), CH₃), 4.69 (2H, s, CH₂), 7.16-7.22 (2H, m, ArH), 7.31 (1H, dt, J 2.5, 7.5 Hz, ArH), 7.45 (1H, d, J 7.5 Hz, ArH); δ_{C} (75 MHz, CDCl₃) −5.4, 18.4, 25.9, 33.6, 61.0, 62.5, 126.4, 126.8, 129.3, 132.7, 138.8, 169.7.

### EXAMPLE 8

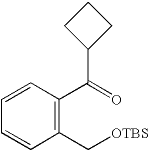

[2-tert-Butyldimethylsilanyloxymethyl)phenyl] cyclobutylmethanone 8:

At −78° C. under argon ^{t}BuLi 1.7 M in pentane (16 cm³, 27.31 mmol, 2 eq.) was added in a dropwise fashion to a solution of cyclobutyl bromide (1.3 cm³, 13.66 mmol, 1 eq.) in THF (15 cm³). The resultant yellow solution was stirred for 1 h at −78° C. before adding via cannula to a cooled (−78° C.) solution of the Weinreb amide 7 (2.11 g, 6.83 mmol, 0.5 eq.) in THF (30 cm³). Stirring was continued for 1 h. Saturated NH₄C₁ solution (50 cm³) was added and the mixture was warmed to room temperature. Extraction with ether (3×50 cm³) and drying of the combined organic extracts over MgSO₄ gave the crude cyclobutane after filtration and solvent removal under reduced pressure. Purification by flash column chromatography (Pet:EtOAc; 8:1→Pet:EtOAc; 5:1) afforded 8 (1.1 g, 59%) as a clear oil. R_{f}=0.55 (Pet:EtOAc; 5:1).

### EXAMPLE 9

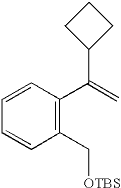

tert-Butyl-[2-(1-cyclobutylvinyl)benzyloxy]dimethylsilane 9:

Under N₂ in foil covered apparatus a mixture of 8 (476 mg, 1.57 mmol, 1 eq.) and Cₚ₂TiMe₂⁷³ (700 mg, 3.35 mmol, 2.1 eq.) in THF (20 cm³) were heated to reflux for 15 h. Petroleum ether (100 cm³) was added and the reaction mixture was filtered through Celite®. The residue was washed with petroleum ether (2×50 cm³) before silica (ca. 5 g) was added and the solvent removed under reduced pressure. Purification by flash column chromatography (Pet:EtOAc; 19:1) gave 9 (434 mg, 92%) as a clear oil. R_{f}=0.25 (Pet:EtOAc; 19:1); m/z (CI) 320 (MNH₄⁺, 5%), 303 (MH⁺, 10%), 171 (100%); found (EI) 302.2062 C₁₉H₃₀OSi requires 302.2066 (−1.3 ppm); δ_{H} (300 MHz, CDCl₃) 0.08 (6H, s, CH₃), 0.84 (9H, s, CH₃), 1.55-1.67 (1H, m, CH₂), 1.69-2.01 (5H, m, CH₂), 3.10 (1H, pent, J 8.0 Hz, CH), 4.58 (2H, s, CH₂), 4.79 (1H, d, J 1.5 Hz, CH₂), 5.06 (1H, d, J 1.5 Hz, CH₂), 6.94 (1H, d, J 7.5 Hz, ArH), 7.09 (1H, t, J 7.5 Hz, ArH), 7.17 (1H, t, J 7.5 Hz, ArH), 7.44 (1H, d, J 7.5 Hz, ArH); δ_{C} (75 MHz, CDCl₃) −5.3, 17.6, 18.4, 25.9, 28.0, 42.0, 62.6, 112.1, 126.2, 126.7, 127.9, 138.0, 140.2, 151.9.

### EXAMPLE 10

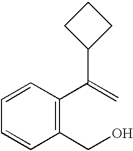

[2-(1-Cyclobutylvinyl)phenyl]methanol 10:

At room temperature a 1 M solution of TBAF (0.9 cm³, 0.90 mmol, 1 eq.) was added dropwise to a solution of 9 (269 mg, 0.89 mmol, 1 eq.) in THF (10 cm³) and stirring was continued for 2 h. Et₂O (15 cm³) and H₂O (25 cm³) were added and the resultant aqueous layer was extracted with Et₂O (2×25 cm³). The combined ethereal extracts were dried over MgSO₄, filtered and the solvent removed under reduced pressure. Purification by flash column chromatography (Pet:EtOAc; 19:1→Pet:EtOAc; 5:1) afforded the title compound 10 (139 mg, 83%) as a clear oil. R_{f}=0.3 (Pet:EtOAc; 5:1); m/z (CI) 206 (MNH₄⁺, 15%), 189 (MH⁺, 10%), 171 (100%); found 189.1277, C₁₃H₁₆O.H requires 189.1279 (−1.1 ppm); δ_{H} (300 MHz, CDCl₃) 1.55-1.67 (1H, m, CH₂), 1.71-2.01 (5H, m, CH₂), 3.13 (1H, pent, J 8.0 Hz, CH), 4.54 (2H, s, CH₂), 4.83 (2H, d, J 1.25 Hz, CH₂), 5.11 (1H, dd, J 1.25 Hz, CH₂), 6.99 (1H, d, J 7.0 Hz, ArH), 7.11-7.22 (2H, m, ArH), 7.36 (1H, d, J 7.25 Hz, ArH); δ_{C} (75 MHz, CDCl₃) 17.6, 27.9, 42.1, 63.2, 112.4, 127.05, 127.1, 128.0, 128.5, 137.7, 141.7, 152.2.

### EXAMPLE 11

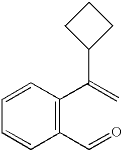

2-(1-Cyclobutylvinyl)benzaldehyde 11:

A solution of 10 (1.90 g, 10.11 mol, 1 eq.) in DCM (60 cm³) was treated with pre-dried MnO₂ (4.40 g, 50.61 mol, 5 eq.) at room temperature and stirring was continued for 2 days. The reaction mixture was then filtered through Celite® and the residue was washed with DCM (2×50 cm³). Concentration in vacuo and purification by flash column chromatography (Pet:EtOAc; 19:1) gave the aldehyde 11 (1.75 g, 93%) as a clear liquid. R_{f}=0.3 (Pet:EtOAc; 19:1); υₘₐₓ (neat, cm⁻¹) 3084, 2976, 2940, 2864, 2748, 1695, 1596, 1479, 1446, 1391; m/z (CI) 264 (MNH₄⁺, 15%), 187 (MH⁺, 60%), 169 (100%); found 187.1121, C₁₃H₁₄O.H requires 187.1122 (−0.5 ppm); □_{H} (300 MHz, CDCl₃) 1.66-1.77 (1H, m, CH₂), 1.79-2.13 (5H, m, CH₂), 3.31 (1H, pent, J 8.0 Hz, CH), 4.96 (1H, d, J 1.5 Hz, CH₂), 5.37 (1H, d, J 1.5 Hz, CH₂), 7.25 (1H, d, J 7.5 Hz, ArH), 7.37 (1H, t, J 7.5 Hz, ArH), 7.52 (1H, t, J 7.5 Hz, ArH), 7.93 (1H, d, J 7.5 Hz, ArH), 10.18 (1H, s, CHO); δ_{C} (75 MHz, CDCl₃) 17.6, 27.7, 42.2, 115.5, 127.2, 127.3, 128.9, 133.2, 133.7, 146.2, 149.2, 192.1.

### EXAMPLE 12

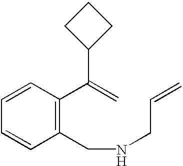

Allyl-[2-(1-cyclobutylvinyl)benzyl]amine 12:

A mixture of aldehyde 11 (540 mg, 2.90 mmol, 1 eq.) and MgSO₄ (ca. 5 g) in DCM (30 cm³) were treated at room temperature with allylamine (0.45 cm³, 6.00 mmol, 2 eq.). The reaction mixture was stirred for 24 h and filtered. Solvent removal gave the crude imine. At room temperature the imine (ca. 2.90 mmol, 1 eq.) was dissolved in MeOH (20 cm³) and NaBH₄ (164 mg, 4.33 mmol, 1.5 eq.) was added portionwise. After stirring for 2 h DCM (50 cm³) and H₂O (50 cm³) were added and the mixture was basified with 2.5 M NaOH (pH 10). The resultant aqueous phase was further extracted with DCM (3×50 cm³) and the combined organics were dried over MgSO₄. Filtration and solvent removal in vacuo gave 127 (600 mg., 91%) as a yellow oil which was used without further purification. m/z (CI) 288 (MH⁺, 100%); δ_{H} (300 MHz, CDCl₃) 1.39-1.51 (1H, s(br), NH), 1.68-1.79 (1H, m, CH₂), 1.87-2.11 (5H, m, CH₂), 3.21 (1H, pent, J 8.0 Hz, CH), 3.25 (2H, dt, J 0.5, 6.5 Hz, CH₂), 3.75 (2H, s, CH₂), 4.93 (1H, s, CH₂), 5.07-5.25 (2H, m, CH₂), 5.19 (1H, s, CH₂), 5.84-6.03 (1H, m, CH), 7.04-7.08 (1H, m, ArH), 7.16-7.29 (2H, m, ArH), 7.39-7.43 (1H, m, ArH); δ_{C} (75 MHz, CDCl₃) 17.6, 28.0, 42.2, 50.6, 51.9, 53.3, 112.0, 115.6, 126.3, 126.8, 128.6, 128.7, 136.9, 137.0, 141.8, 152.7.

### EXAMPLE 13

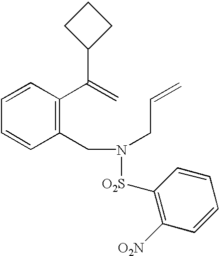

N-Allyl-N-[2-(1-cyclobutylvinyl)benzyl]-2-nitrophenylsulfonamide 13:

A mixture of the amine 12 (200 mg, 0.881 mmol, 1 eq.), TEA (0.18 cm³, 1.291 mmol, 1.5 eq.), 2-NsCl (215 mg, 0.970 mmol, 1.1 eq.) and a catalytic amount of DMAP (ca. 2 mg) in DCM (10 cm³) were stirred at room temperature for 3 h. Et₂O (25 cm³) and H₂O (25 cm³) were added and the resultant aqueous layer was further extracted with Et₂₀ (2×15 cm³). The combined organic extracts were dried over MgSO₄. Filtration, solvent removal under reduced pressure followed by flash column chromatography (Pet:EtOAc; 3:1) gave the title compound 13 (285 mg, 79%) as a clear viscous oil. R_{f}=0.3 (Pet:EtOAc; 3:1); m/z (CI) 430 (MNH₄⁺, 15%), 413 (MH⁺, 5%), 383 (30%), 228 (50%), 171 (100%); found 413.1528,C₂₂H₂₄N₂O₄S.H requires 413.1535 (−1.7 ppm); δ_{H} (300 MHz, CDCl₃) 1.63-1.78 (1H, m, CH₂), 1.82-2.11 (5H, m, CH₂), 3.16 (1H, pent, J 8.25 Hz, CH), 3.94 (2H, d, J 6.25 Hz, CH₂), 4.61 (2H, s, CH₂), 4.88 (1H, s, CH₂), 5.01-5.12 (2H, m, CH₂), 5.22 (1H, t(br), J 1.5 Hz, CH₂), 5.61 (1H, tq, J 6.25 Hz, CH), 7.04-7.11 (1H, m, ArH), 7.18-7.26 (2H, m, ArH), 7.35-7.41 (1H, m, ArH), 7.63-7.76 (3H, m, ArH), 8.04 (1H, d, J 7.5 Hz, ArH); δ_{C} (75 MHz, CDCl₃) 17.6, 28.0, 42.0, 48.1, 49.6, 112.8, 119.0, 124.1, 126.8, 127.1, 127.2, 128.6, 131.0, 131.6, 132.0, 132.2, 133.4, 134.0, 141.8, 143.8, 151.6.

### EXAMPLE 14

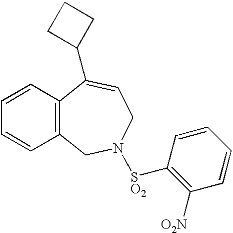

5-Cyclobutyl-2-(2-nitrophenylsulfonyl)-2,3-dihydro-1H-benzo[c]azepine 14:

The dialkenyl sulfonamide 13 (885 mg, 2.12 mmol, 1 eq.) and Grubbs catalyst (90 mg, 0.106 mmol, 5 mol %) in degassed DCM (100 cm³) were heated to reflux for 18 h. The Grubb's catalyst used was tricyclohexylphosphine-[1,3-bis(2,4,6-trimethylphenyl)-4,5-dihydroimidazol-2-ylidene][benzylidine]ruthenium(IV)dichloride, available from Strum Chemicals Inc., Catalogue No. 77-7770. The reaction mixture was cooled to room temperature and silica (ca. 3 g) was added. Solvent removal under reduced pressure and purification by flash column chromatography (Pet:EtOAc; 3:1) gave the title compound 14 (780 mg, 96%) as a clear viscous oil. R_{f}=0.25 (Pet:EtOAc; 3:1); m/z (CI) 385 (MH⁺, 5%), 355 (20%), 198 (90%), 94 (100%); found 385.1224, C₂₀H₂₀N₂O₄S.H requires 385.1222 (+0.5 ppm); δ_{H} (300 MHz, CDCl₃) 1.72-1.82 (1H, m, CH₂), 1.83-2.04 (3H, m, CH₂), 2.13-2.24 (2H, m, CH₂), 3.49 (1H, pent, J 8.0 Hz, CH), 3.67 (2H, d, J 7.5 Hz, CH₂), 4.19 (2H, s, CH₂), 5.90 (1H, dt, J 2.0, 7.5 Hz, CH), 7.25-7.32 (2H, m, ArH), 7.35-7.42 (2H, m, ArH), 7.64-7.77 (3H, m, ArH), 8.05 (1H, dd, J 2.0, 5.5 Hz, ArH); δ_{C} (75 MHz, CDCl₃) 17.8, 28.4, 39.5, 43.0, 49.2, 116.9, 124.0, 126.1, 128.0, 129.8, 130.5, 131.5, 132.9, 133.2, 133.3, 139.9, 148.2, 151.0.

### EXAMPLE 15a

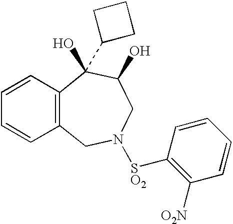

5-Cyclobutyl-2-(2-nitrophenylsulfonyl)-2,3,4,5-tetrahydro-1H-benzo[c]azepine-4,5-diol 15:

At room temperature the alkene 14 (120 mg, 0.313 mmol, 1 eq.) was dissolved in acetone (5 cm³) and H₂O (2.5 cm³) and NMO (40 mg, 0.341 mmol, 1.1 eq.) were added. OsO₄ (8 mg, 0.0315 mmol, 10 mol %) was then added to the vigourously stirred mixture. Stirring was continued for 18 h. DCM (15 cm³) and H₂O (15 cm³) were added and the mixture was acidified with 3 M HCl (pH 2). The aqueous phase was further extracted with DCM (3×15 cm³) and the combined organics were dried over MgSO₄. Filtration, solvent removal in vacuo followed by flash column chromatography (Pet:EtOAc; 2:1) gave 15 (104 mg, 80%) as an amorphous grey solid. The diol 15 was further purified by reprecipitation from Et₂O and petroleum ether. R_{f}=0.3 (Pet:EtOAc; 1:1); υₘₐₓ (CDCl₃, cm⁻¹) 3540, 3093, 2982, 2940, 2867, 1590, 1545, 1445; 1371, 1352, 1164; m/z (CI) 436 (MNH₄⁺, 10%), 419 (MH⁺, 5%), 389 (20%), 232 (40%), 94 (100%); found 419.1274, C₂₀H₂₂N₂O₆S.H requires 419.1277 (−0.7 ppm); δ_{H} (300 MHz, CDCl₃) 1.29. (1H, m, CH₂), 1.78-1.90 (3H, m, CH₂), 2.13-2.38 (2H, m, CH₂), 2.49 (1H, d, J 9.25 Hz, OH), 2.91 (1H, pent, J 8.0 Hz, CH), 3-22 (1H, s, OH), 3.53 (1H, dd, J 1.0, 15.0 Hz, CH₂), 3.86 (1H, m, CH), 4.03 (1H, ddd, J 2.0, 4.0, 15.0 Hz, CH₂), 4.46 (1H, d, J 16.0 Hz, CH₂), 4.83 (1H, dd, J 2.0, 16.0 Hz, CH₂), 7.23-7.26 (2H, m, ArH), 7.36-7.40 (1H, m, ArH), 7.67-7.81 (3H, m, ArH), 7.86 (1H, d, J 7.5 Hz), 8.11 (1H, dd, J 2.0, 7.0 Hz, ArH); δ_{C} (75 MHz, CDCl₃) 17.5, 21.6, 21.8, 39.3, 50.9, 54.2, 72.5, 79.2, 124.2, 127.6, 128.2, 129.2, 130.2, 131.4, 131.7, 132.2, 132.8, 133.9, 140.8, 147.9.

### EXAMPLE 15b

5-Cyclobutyl-2-(2-nitrophenylsulfonyl)-2,3,4,5-tetrahydro-1H-benzo[c]azepine-4R,5R-diol 15:

At 5° C. a mixture of dihydroazepine 14 (96 mg, 0.25 mmol, 1 eq.), AD-mix-β (180 mg) and MeSO₂NH₂ (24 mg, 0.25 mmol, 1 eq.) in ^{t}BuOH (1 cm³) and H₂O (1 cm³) were stirred for 2 days. Saturated Na₂SO₃ (5 cm³) and DCM (10 cm³) were added and the mixture was partitioned vigorously for 1 h. The resultant aqueous phase was further extracted with DCM (3×10 cm³) and the combined organic phases were dried over MgSO₄. Filtration, solvent removal and purification by flash column chromatography (Pet:EtOAc; 1:1) afforded 14 (41 mg, 43%) and the diol 15 (52 mg, 50%) whose data was in agreement to that reported above.

### EXAMPLE 16

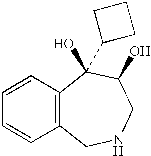

5-Cyclobutyl-2,3,4,5-tetrahydrobenzo[c]azepine-4,5-diol 16:

At room temperature a mixture of nosylate 15 (256 mg, 0.61 mmol, 1 eq.) and K₂CO₃ (296 mg, 2.14 mmol, 3.5 eq.) in DMF (15 cm³) was treated with PhSH (80 μL, 0.78 mmol, 1.3 eq.). Stirring was continued at room temperature for 24 h. Ethyl acetate (25 cm³) and water (25 cm³) was added and the resultant aqueous layer was further extracted with EtOAc (5×25 cm³). The combined organic extracts were dried over MgSO₄. Filtration followed by solvent removal and column chromatography (EtOAc:MeOH; 3:1, 1% TEA) afforded the diol 16 (122 mg, 86%) as an amorphous colourless solid. R_{f}=0.25 (EtOAc:MeOH; 3:1, 1% TEA); m/z (CI) 234 (MH⁺, 100%); found 234.1498, C₁₄H₁₉NO₂.H requires 234.1494 (+1.7 ppm); δ_{H} (300 MHz) 1.26-1.38 (1H, m, CH₂), 1.69-1.88 (3H, m, CH₂), 2.07-2.28 (2H, m, CH₂), 2.81-2.94 (1H, m, CH), 3.05 (1H, d, J 13.5 Hz, CH₂), 3.18 (1H, dd, J 3.5, 13.5 Hz, CH₂), 3.66 (1H, d, J 3.5 Hz, CH), 3.84 (1H, d, J 15.0 Hz, CH₂), 4.00 (1H, d, J 15.0 Hz, CH₂), 7.01 (1H, d, J 7.5 Hz, ArH), 7.16 (1H, t, J 7.5 Hz, ArH), 7.28 (1H, t, J 7.5 Hz, ArH), 7.80 (1H, d, J 7.5 Hz, ArH); δ_{C} (75 MHz) 17.6, 21.6, 21.8, 39.7, 51.9, 55.8, 73.2, 79.8, 126.9, 127.1, 129.2, 129.5, 136.3, 141.7.

### EXAMPLE 17

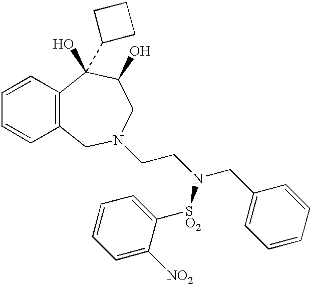

N-Benzyl-N-[2-(5-cyclobutyl-4,5-dihydroxy-1,3,4,5-tetrahydrobenzo [c]azepin-2-yl)ethyl]-2-nitro-benzenesulfonamide 17:

At room temperature 16 (122 mg, 0.524 mmol, 1 eq.) and the aldehyde 6 (see Example 6) (350 mg, 1.048 mmol, 2 eq.) in MeOH (5 cm) were treated with NaBH₃CN (33 mg, 0.525 mmol, 1 eq.) and conc. HCl (1 drop). The mixture was stirred for 15 h before EtOAc (25 cm³) and H₂O (25 cm³) were added. The pH was adjusted with 1 M NaOH to ca. 12 and the resultant aqueous layer was further extracted with EtOAc (2×25 cm³) and DCM (3×25 cm³). The combined organic extracts were dried over MgSO₄ and filtered. Silica (ca. 2.5 g) was added and the solvent was removed in vacuo. Purification by flash column chromatography (Pet:EtOAc; 1:1; 1% TEA→EtOAc; 1% TEA) gave the adduct 17 (189 mg, 66%) as a viscous yellow oil. R_{f}=0.2 (Pet:EtOAc; 1:1); ); υₘₐₓ (CDCl₃/cm⁻¹) 3468, 3065, 2940, 2867, 1544, 1455, 1370, 1162; m/z (CI) 552 (MH⁺, 2%), 363 (10%), 108 (100%); found 552.2172, C₂₉H₃₃N₃O₆S.H requires 552.2168 (+0.7 ppm); δ_{H} (300 MHz, CDCl₃) 1.19-1.31 (1H, m, CH₂), 1.57-1.77 (3H, m, CH₂), 2.05-2.22 (2H, m, CH₂), 2.50-2.62 (2H, m, 25 CH₂), 2.71 (1H, pent, J 8.5 Hz, CH), 2.82-2.93 (2H, m, CH₂), 3.31 (1H, ddd, J 5.0, 8.5, 15.0 Hz, CH₂), 3.38 (1H, dd, J 5.5, 15.0 Hz, CH₂), 3.44 (1H, dd, J 7.5, 15.0 Hz, CH₂), 3.58 (1H, d, J 3.5 Hz, CH), 3.69 (1H, d, J 15.0 Hz, CH₂), 6.91 (1H, d, J 7.5 Hz, ArH), 7.10 (1H, dt, J 1.5, 7.5 Hz, ArH), 7.18-7.31 (6H, m, ArH), 7.53-7.67 (3H, m, ArH), 7.73 (1H, dd, J 1.5, 8.0 Hz, ArH), 7.95 (1H, d, J 8.0 Hz, ArH); δ_{C} (100 MHz, CDCl₃) 17.7, 21.7, 21.8 (CH₂), 39.6 (CH), 45.5, 52.2, 57.5, 60.2, 63.0 (CH₂), 73.1 (CH), 79.3 (C-quat), 124.2, 126.8, 127.2, 128.1, 128.15, 128.7, 128.8, 130.2, 130.8, 131.6 (CH), 133.1 (C-ipso), 133.4 (CH), 134.5, 135.1, 141.4, 147.8 (C-ipso).

### EXAMPLE 18

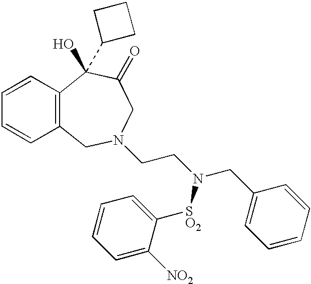

N-Benzyl-N-[2-(5-cyclobutyl-5-hydroxy-4-oxo-1,3,4,5-tetrahydrobenzo[c]azepin-2-yl)ethyl]-2-nitro-benzenesulfonamide 18:

A solution of (COCl)₂ (42 μL, 0.481 mmol, 4 eq.) in DCM (1 cm³) was treated at −78° C. with DMSO (60 μL, 0.846 mmol, 7 eq.). Stirring was continued for 0.25 h before a solution of 17 (60 mg, 0.109 mmol, 1 eq.) in DCM (0.1 cm³) was added in a dropwise fashion. Additionally, the flask was washed with DCM (1 cm³). The reaction mixture was stirred for 2 h during which time the temperature reached −10° C. TEA (100 □L, 0.718 mmol, 6 eq.) was added to the reaction mixture. Stirring was continued for×h before H₂O (20 cm³) and Et₂O (20 cm³) were added. The resultant aqueous layer was further extracted with Et₂O (4×20 cm³) and the combined organic extracts were dried over MgSO₄. Filtration followed by solvent removal and purification by flash column chromatography (Pet:EtOAc; 3:1, 1% TEA→Pet:EtOAc; 1:1, 1% TEA) afforded 18 (46 mg, 77%) as a yellow oil. R_{f}=0.2 (Pet:EtOAc; 3:1, 1% TEA); ); υₘₐₓ (neat/cm⁻¹) 3466, 3065, 2936, 2861, 1698, 1544, 1454, 1369, 1163; m/z (CI) 550 (MH⁺, 20%), 363 (40%), 106 (90%), 94 (100%); found 550.2021, C₂₉H₃₁N₃O₆S.H requires 550.2012 (+1.6 ppm); δ_{H} (300 MHz, CDCl₃) 1.49-1.62 (1H, m, CH₂), 1.65-1.88 (4H, m, CH₂), 2.22 (1H, pent, J 9.0 Hz, CH), 2.33 (2H, t, J 7.0 Hz, CH₂), 3.34-3.39 (3H, m, CH, CH₂), 3.38 (1H, d, J 15.0 Hz, CH₂), 3.64 (1H, d, J 15.0 Hz, CH₂), 3.76 (1H, d, J 16.0 Hz, CH₂), 4.04 (1H, d, J 16.0 Hz, CH₂), 4.50 (2H, s, CH₂), 4.54 (1H, s(br), OH), 6.91 (1H, d, J 7.5 Hz, ArH), 7.10 (1H, dt, J 1.5, 7.5 Hz, ArH), 7.18-7.31 (6H, m, ArH), 7.53-7.67 (3H, m, ArH), 7.73 (1H, dd, J 1.5, 8.0 Hz, ArH), 7.95 (1H, dd, J 1.6, 8.0 Hz, ArH); δ_{C} (75 MHz, CDCl₃) 17.0, 21.3, 21.6, 41.6, 44.8, 51.0, 52.1, 59.5, 63.5, 84.1, 124.2, 127.3, 127.5, 127.6, 128.1, 128.2, 128.7, 129.9, 130.8, 131.6, 133.4, 133.6, 133.8, 135.4, 138.3, 148.1, 206.8.

### EXAMPLE 19

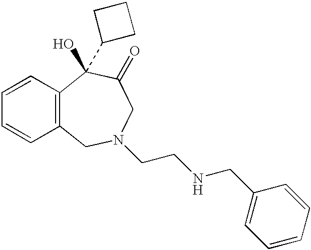

2-[2-(benzylamino) ethyl]-5-cyclobutyl-5-hydroxy-1,3,4,5-tetrahydrobenzo[c]azepin-4-one 19:

At room temperature a solution 18 (260 mg, 0.474 mmol, 1 eq.) in DMF (5 cm³) was treated with K₂CO₃ (212 mg, 1.534 mmol, 3.2 eq.) and PhSH (60 μL, 0.584 mmol, 1.2 eq.). Stirring was continued for 18 h before H₂O (25 cm³) and EtOAc (25 cm³) were added. The resultant aqueous layer was extracted with EtOAc (5×15 cm³) and the combined organic layers were dried over MgSO₄. The crude amine obtained after filtration and solvent removal in vacuo was purified by flash column chromatography (Pet:EtOAc; 1:1, 1% TEA→Pet:EtOAc; 1:2, 1% TEA) which gave the title compound 19 (90 mg, 52%) as a yellow oil. R_{f}=0.15 (Pet:EtOAc; 1:1, 1% TEA); υₘₐₓ (neat/cm⁻¹) 3454, 3054, 2934, 2855, 1692, 1453; m/z (CI) 365 (MH⁺, 80%), 347 (M-OH⁺, 30%), 108 (100%), 74 (80%); found 365.2220, C₂₃H₂₈N₂O₂.H requires 365.2229 (−2.5 ppm); δ_{H} (300 MHz, CDCl₃) 1.40-1.48 (1H, m, CH₂), 1.58-1.76 (4H, m, CH₂), 2.08-2.16 (1H, m, CH₂), 2.48 (2H, t, J 6.0 Hz, CH₂), 2.71 (2H, t, J 6.0 Hz, CH₂), 3.26 (1H, pent, J 8.5 Hz, CH), 3.44 (1H, d, J 15.5 Hz, CH₂), 3.75 (2H, d, J 15.5 Hz, CH₂), 3.76 (1H, d, J 16.5 Hz, CH₂), 3.90 (1H, d, J 13.5 Hz, CH₂), 3.96 (1H, d, J 13.5 Hz, CH₂), 4.15 (1H, d, J 16.5 Hz, CH₂), 6.84 (1H, d, J 7.5 Hz, ArH), 7.05 (1H, dt, J 1.0, 7.5 Hz, ArH), 7.11-7.36 (6H, m, ArH), 7.64 (1H, d, J 7.5 Hz, ArH).

### EXAMPLE 20

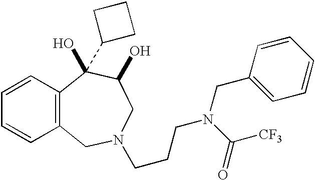

N-Benzyl-N-[3-(5-cyclobutyl-4,5-dihydroxy-1,3,4,5-tetrahydrobenzo[c]azepin-2-yl)propyl]-2,2,2-trifluoroacetamide 20:

At room temperature the nosylate 15 (120 mg, 0.287 mmol, 1 eq.) and K₂CO₃ (129 mg, 0.933 mmol, 3.25 eq.) in DMF (5 cm³) was treated with phenylmercaptan (44 □L, 0.428 mmol, 1.49 eq.). Stirring was continued for 24 h before the reaction mixture was exhaustively extracted with EtOAc (5×25 cm³) and H₂O (25 cm³). The combined organic extracts were dried over MgSO₄, filtration followed by solvent removal in vacuo afforded the crude diol 16. A mixture of the crude diol 16 (ca. 0.287 mmol, 1 eq.) the aldehyde 3 (223 mg, 0.861 mmol, 3 eq.), NaBH₃CN (18 mg, 0.286 mmol, 0.99 eq.) in MeOH (5 cm³) with a drop of conc. HCl were stirred at room temperature for 15 h. The reaction mixture was extracted with Et₂O (5×25 cm³) and 1 M NaOH (25 cm³), dried over MgSO₄. Filtration followed by solvent removal under reduced pressure afforded the crude adduct which was purified by column chromatography (Pet:EtOAc; 3:1, 1% TEA→Pet:EtOAc; 1:1, 1% TEA) gave the title compound 20 (104 mg, 76%). R_{f}=0.1 (Pet:EtOAc; 3:1, 1% TEA); 0.3 (Pet:EtOAc; 1:1, 1% TEA);.m/z (CI) 477 (MH⁺, 100%); found (EI) 476.2287, C₂₆H₃₁N₂O₃F₃ requires 476.2290 (−0.8 ppm); δ_{H} (300 MHz, CDCl₃) 1.28-1.45 (1H, m, CH₂), 1.63-1.91 (3H, m, CH₂), 2.14-2.36 (2H, m, CH₂), 2.45-2.59 (2H, m, CH₂), 2.61-2.72 (2H, m, CH₂), 2.75-2.96 (2H, m, CH_{A}H_{B}, CH), 2.98-3.05 (1H, m, CH_{A}H_{B}), 3.22-3.42 (2H, m, CH₂), 3.50-3.61 (1H, m, CH_{A′}H_{B′}), 3.62-3.75 (1H, m, CH), 3.75-3.82 (1H, m, CH_{A′}H_{B′}), 4.52\hat{ } (1H, d, J 14.5 Hz, CH₂), 4.56* (1H, d, J 15.5 Hz, CH₂), 4.69* (1H, d, J 15.5 Hz, CH₂), 4.82\hat{ } (1H, d, J 14.5 Hz, CH₂), 7.05-7.18 (2H, m, ArH), 7.19-7.25 (1H, m, ArH), 7.25-7.45 (5H, m, ArH), 7.80-7.88 (1H, m, ArH). ¹H-NMR complicated due to rotameric structures [60*:40\hat{ }].

### EXAMPLE 21

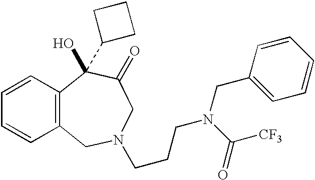

N-Benzyl-N-[3-(5-cyclobutyl-5-hydroxy-4-oxo-1,3,4,5-tetrahydrobenzo[c]azepin-2-yl)propyl]-2,2,2-trifluoroacetamide 21:

A solution of (COCl)₂ (98 μL, 1.12 mmol, 3 eq.) in DCM (3 cm³) was treated at −78° C. with DMSO (133 μL, 1.87 mmol, 5 eq.). Stirring was continued for 10 min. before a solution of 20 (179 mg, 0.376 mmol, 1 eq.) in DCM (3 cm³) was added in a dropwise fashion. This flask was washed with DCM (2 cm³) and this was also added. The reaction mixture was stirred for 1 h during which time the temperature reached 0° C. TEA (0.31 cm³, 2.22 mmol, 6 eq.) was added to the reaction mixture and stirring was continued for 0.5 h. H₂O (25 cm³) and Et₂O (25 cm³) were added and the resultant aqueous layer was further extracted with Et₂O (4×25 cm³) and the combined organic extracts were dried over MgSO₄. Filtration followed by solvent removal and purification by flash column chromatography (Pet:EtOAc; 5:1, 1% TEA) afforded 21 (120 mg, 67%) as a yellow oil. R_{f}=0.3 (Pet:EtOAc; 5:1, 1% TEA); υₘₐₓ (neat/cm⁻¹) 3466, 3065, 2942, 2861, 1691, 1452, 1376, 1202, 1144; m/z (CI) 475 (MH⁺, 5%), 261 (40%), 221 (80%), 108 (100%), 91 (90%); found 475.2205, C₂₆H₂₉N₂O₃F₃.H requires 475.2208 (−0.6 ppm); δ_{H} (200 MHz, CDCl₃) 1.49-1.98 (8H, m, CH₂), 2.18-2.40 (2H, m, CH₂), 3.28-3.60 (4H, m, CH₂, CH_{A}H_{B}, CH), 3.74-3.90 (2H, m, CH_{A}H_{B}, CH_{A′}H_{B′}), 4.15-4.31 (1H, m, CH_{A′}H_{B′}), 4.55-4.73 (2H, m, CH₂), 6.95-7.01 (1H, m, ArH), 7.10-7.45 (7H, m, ArH), 7.71-7.83 (1H, m, ArH). ¹H-NMR complicated due to rotameric structures.

### EXAMPLE 22

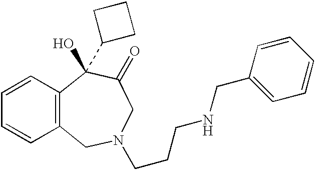

2-[3-(Benzylamino) propyl]-5-cyclobutyl-5-hydroxy-1,2,3,5-tetrahydrobenzo[c]azepin-4-one 22:

At room temperature a solution of 21 (80 mg, 0.169 mmol, 1 eq.) in MeOH (10 cm³) was treated with a solution of K₂CO₃ (117 mg, 0.847 mmol, 5 eq.) in H₂O (0.6 cm³). Stirring was maintained for 24 h. Solvent removal in vacuo followed by purification by flash column chromatography (Pet:EtOAc; 1:2, 1% TEA) gave the title compound 22 (50 mg, 80%) as a yellow oil. R_{f}=0.2 (Pet:EtOAc; 1:2); m/z (CI) 379 (MH⁺, 50%), 284 (50%), 267 (55%), 108 (100%); found 379.2381, C₂₄H₃₀N₂O₂.H requires 379.2385 (−1.1 ppm); δ_{H} (300 MHz, CDCl₃) 1.41-1.88 (7H, m, CH₂), 2.13-2.28 (1H, m, CH₂), 2.38 (2H, dt, J 3.5, 7.0 Hz, CH₂), 2.59 (2H, t, J 7.0 Hz, CH₂), 3.39-3.51 (1H, m, CH), 3.45 (1H, d, J 15.75 Hz, CH₂), 3.68 (1H, d, J 15.75 Hz, CH₂), 3.69 (2H, s, CH₂), 3.85 (1H, d, J 16.25 Hz, CH₂), 4.14 (1H, d, J 16.25 Hz, CH₂), 6.97 (1H, d, J 7.5 Hz, ArH), 7.11 (1H, dt, J 1.5, 7.5 Hz, ArH), 7.14-7.26 (6H, m, ArH), 7.67 (1H, d, J 7.5 Hz, ArH); δ_{C} (75 MHz, CDCl₃) 17.2, 21.6, 21.7, 27.4, 30.3, 41.7, 47.1, 52.5, 60.1, 64.1, 84.0, 127.0, 127.3, 127.4, 128.0, 128.4, 129.6, 134.5, 138.4, 207.7.

Pharmacology

Functional Assays of M1, M2 and M3 Receptor Activity

Initial evaluation of test compounds is by assay of functional tissue responses. This has the advantage that it readily discriminates between agonist partial agonist and antagonist activity

M1—Vas Deferens Preparations

Male New Zealand white rabbits (1.47-3.4 kg) are killed by a blow to the back of the head and vasa deferentia removed, dissected free of connective tissue and divided into prostatic and epididymal portions. Each segment is mounted on a tissue holder and passed through two ring electrodes (5 mm apart). They are immersed in a modified low Ca2+ Krebs solution at 32?0.51C and gassed with 5% CO₂ in oxygen. Yohimibine (11.0 mM) is present throughout to block prejunctional a2-adrenoceptros. The upper end of the tissue is attached by cotton thread to an isometric transducer (MLT020, ADInstruments). Tissues are left to equilibrate for at least 45 min at passive force of 0.75-1 g. Field stimulation is then applied by repeated application of single pulses (30V, 0.05 Hz, 0.5 ms). Isometric tension is recorded by computer at a sampling rate of 100 HZ, using Powerlab/200 (ADInstruments) software and MacLab bridge amplifiers.

M2—Guinea-Pig Atria

Guinea-pigs are killed by a blow to the back of the head and left atrium removed. The atrium is secured to a pari of stainless steel electrodes by means of a cotton thread and immersed in the organ bath containing gassed Krebs solution with normal Ca^{2 +} at 32±0.50C. Atria are placed at 2 Hz with square-wave pulses of 0.5 ms pulse width. Isometric contractions are recorded by computer or polygraph.

M3—Guinea-Pig Ileum

Sections (2 cm) are cut from the ileum of the killed guinea-pigs, 10 cm from the ileo-caecal junction. One end is attached to a tissue holder/aerator and the other end via a cotton thread to an isometric transducer. The tissue is immersed in gassed normal Ca²⁺ Krebs solution at 32±0.5° C. A resting tension of 0.5 g is applied and isometric contractions measured by computer or polygraph.

Agonist Concentration-Response Curves

Following at least 30 min equilibration to allow twitches or tension to stabilize, cumulative concentration-response curves for the muscarinic agonists are constructed. The concentration is increased in half logarithmic increments after the contraction in the presence of each concentration has plateaued Steady-state contractions at each concentration are measured and the inhibition expressed as a percentage of the baseline twitch height in atria and vas deferens or as the maxi contraction in the ileum. EC50 values for the muscarinic agonists are determined from individual curves as the molar concentration required for 50% inhibition of twitch height or the 50% of maximum contraction (ileum). Geometric mean EC50 values and their 95% confidence limits are calculated.

Effects of Muscarinic Antagonists

A concentration-response curve to the test agonist is established in the absence of antagonist and after achieving the maximum effect, the agonist washed from the bath to restore twitch contractions. Three further concentration-response curves are then obtained in the same manner at approximately 30 min intervals, with the antagonist (Standards—pirenzepine M1, darifenacin M3, methoctramine M2) being introduced to the bath 15 min before each of these subsequent curves.

Calculation of Antagonist pA₂ Values

Concentration-response curves in the absence and presence of antagonist are measured as described for the agonist studies. The shifts in the concentration-response curves in the presence of antagonist compared with the absence of antagonist are expressed as the dose-ratios (DR) of the EC50 values. pA₂ values are then determined from Schild analysis of plots of the mean corrected −log(DR−1) against log molar concentration of antagonist. The slopes of the Schild plots are determined by linear regression and the pA₂ values determined from the intercept on the concentration axis (when log(DR−1) is zero). pA₂ values are also determined from individual concentrations of antagonist by applying the equations: pA₂=log(DR−1) log[B], wherein B is the molar concentration of antagonist.

Standard Drugs

Carbamoylcholine chloride (carbachol), methacholine, methactramine, pirenzepine dihydrochloride, yohimbine hydrochloride (Sigma, Poole, Dorset, UK), darifenacin (Pfizer, Sandwich, Kent), McN-A343 [4-(4-chlorophenylcarbamoyloxy)-2-butynyl-trimethylammonium iodide] and oxotremorine sesquifurnarate (RBI, St. Albans, UK). AR drugs are dissolved in distilled water initially and dilutions made in Krebs solution.

Reference Data

The reproducibility of the concentration-response curves and stability of each tissue over several hours was established. EC50 values were obtained for a range of reference agonists in each tissue—methacholine, oxotremorine, McN-A-343 to permit comparisons with the novel agents of the invention. pA₂ values for reference antagonists were obtained in relevant tissues—pirenzepine (M1 selective), darifenacin (M3 selective). It is as a result possible to establish the functional characterization of the three receptor types to enable determination of the potency (EC50 of agonist, pA₂ or affinity of antagonist molecules) and selectively of the novel agents of the invention.

The compounds of Examples 19 and 22 were tested as described above and the results obtained were as follows:

| Compound | M3 (ileum) | M3 (Atria) | Log₁₀ selectivity |
|---|---|---|---|
| Compound 22 | 6.7 ± 0.4 (4 pts) | 5.2 ± 0.3 (4 pts) | 1.5 ± 0.5 |
| (Example 22) | | | |
| Compound 19 | 6.5 ± 0.4 (3 pts) | 4.9 ± 0.6 (4 pts) | 1.6 ± 0.7 |
| (Example 19) | | | |

1. A compound having the formula: 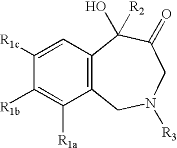
wherein:
R₁ₐi R_{1b} and R_{1c} are independently fluorine or hydrogen;
R₂ is C₁ to C₁₂ alkyl, said alkyl being straight or branched chain, saturated or unsaturated, mono-substituted or unsubstituted, said substituents being selected from piperidine, pyrrolidine, morpholine, thiomorpholine, tetrahydrofuran, thiophen, furan and cycloalkyl of 3 to 7 carbon atoms; a cycloalkyl of 3 to 9 carbon atoms; a cycloalkyl of 3 to 9 carbon atoms (preferably 4 to 9 carbon atoms) having a C₁ to C₆ alkyl substituent; a polycycloalkyl of 2 to 3 rings having 7 to 12 carbons; and phenyl or phenyl singly or multiply substituted (preferably singly or doubly) with halogen, hydroxy, C₁ to C₆ alkoxy, C₁ to C₆ alkyl, nitro, methylene dioxy or trifluoromethyl; and
R₃ is a moiety selected from: 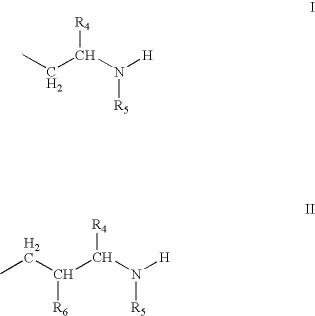
or a pyrrolidin-3-yl moiety of the formula 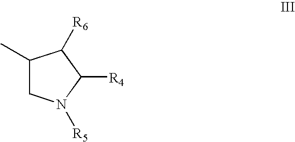
where R₆ is hydroxy or hydrogen;
where one of R₄ and R₅ is hydrogen or lower C1-3 alkyl and the other is selected from:
(a) hydrogen,
(b) phenyl,
(c) phenyl singly or multiply substituted with halogen, hydroxy, C₁ to C₆ alkoxy, C₃ to C₆ alkyl, nitro, methylene dioxy or trifluoromethyl,
(d) C₁ to C₆ alkyl which may be branched chain or straight, saturated, unsaturated, or cyclic and may be optionally substituted with hydroxy, thienyl, pyrrolyl, pyridyl, furanyl, lower alkoxy or acetoxyalkyl wherein the alkyl group has 1 to 3 carbons, phenyl, phenyl singly or multiply substituted (preferably singly or doubly) with halogen, hydroxy, C₁ to C₁₀ alkoxy, C₁ to C₁₀ alkyl, nitro, methylene dioxy (optionaly mono or dialkyl substituted where the alkyl substituent has from 1 to 10 carbon atoms) or trifluoromethyl;
or a pharmaceutically acceptable salt thereof

2. A compound according to claim 1, wherein R₁ is cycloalkyl of 3 to 6 carbon atoms.

3. A compound according to claim 2, wherein R₂ is cyclobutyl.

4. A compound according to any preceding claim, wherein R₄ is hydrogen and R₅ is selected from amongst the groups (a)-(d) as defined in claim 1.

5. A compound according to any one of claims 1 to 3, wherein one of R₄ and R₅ is hydrogen (or methyl in the case of RS) and the other is selected from hydrogen, C₁ to C₆ alkyl which may be branched chain or straight, saturated, unsaturated, or cyclic and may be optionally substituted with hydroxy, thienyl, pyrrolyl, pyridyl, furanyl, phenyl, phenyl singly or multiply substituted (preferably singly or doubly) with halogen, hydroxy, C₁ to C₁₀ alkoxy, C₁ to C₁₀ alkyl or nitro.

6. A compound according to claim 5, wherein R₄ is hydrogen and R₅ is C₁ to C₆ alkyl substituted by phenyl or phenyl which is singly or multiply substituted with halogen, hydroxy, C₁ to C₁₀ alkoxy, C₁ to C₁₀ alkyl or nitro.

7. A compound according to claim 6, wherein R₅ is benzyl, substituted benzyl or cinnamyl.

8. A compound according to claim 7, wherein R₅ is substituted benzyl in which the substituent(s) on the benzyl are independently halo, C₁ to C₁₀alkoxy or C₁ to C₁₀alkyl.

9. A compound according to any preceding claim, wherein R₆ is hydrogen.

10. A compound according to claim 1, wherein R₁ₐ, R_{1b} and R_{1c} are independently hydrogen or fluorine, R₂ is cycloalkyl of 3 to 6 carbon atoms or phenyl, R₃ is 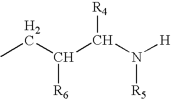
where R₄ is hydrogen and R₅ is selected from C₁ to C₆ alkyl, benzyl, substituted benzyl or cinnamyl, and R₆ is hydrogen or hydroxy.

11. A compound according to claim 10, wherein R₂ is cyclobutyl and R₅ is substituted benzyl in which the substituent(s) on the benzyl are independently halo, C₁ to C₁₀alkoxy or C₁ to C₁₀ alkyl.

12. A compound according to claim 10 or 11, wherein R₆ is hydrogen.

13. A compound according to claim 1, wherein R₁ₐ, R_{1b} and R_{1c} are independently hydrogen or fluorine, R₂ is cycloalkyl of 3 to 6 carbon atoms or phenyl, R₃ is 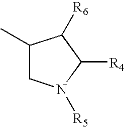
where R₄ is hydrogen and R₅ is selected from C₁ to C₆ alkyl, benzyl, substituted benzyl or cinnamyl, and R₆ is hydroxy or hydrogen.

14. A compound according to claim 13, wherein R₂ is cyclobutyl and R₅ is substituted benzyl in which the substituent(s) on the benzyl are independently halo, C₁ to C₁₀alkoxy or C₁ to C₁₀ alkyl.

15. A compound according to claim 13 or 14, wherein R₆ is hydrogen.

16. A compound according to claim 15, wherein R₁ₐ, R_{1b} and R_{1c} are independently hydrogen or fluorine, R₂ is cycloalkyl of 3 to 6 carbon atoms or phenyl, R₃ is a moiety having the following structure: 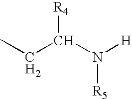
where R₄ is hydrogen and R₅ is selected from C₁ to C₆ alkyl, benzyl, substituted benzyl or cinnamyl.

17. A compound according to claim 16, wherein R₂ is cyclobutyl and R₅ is substituted benzyl in which the substituent(s) on the benzyl are independently halo, C₁ to C₁₀ alkoxy or C₁ to C₁₀ alkyl.

18. A compound according to any preceding claim, wherein R₁ₐ, R_{1b} and R_{1c} are each hydrogen.

19. A compound according to claim 1, which is 2-[2-(benzylamino)ethyl]-5-cyclobutyl-5-hydroxy-1,3,4,5-tetrahydrobenzo[c]azepin-4-one, or a pharmaceuticaly acceptable salt thereof.

20. A compound according to claim 1, which is 2-[3-(Benzylamino)propyl]-5-cyclobutyl-5-hydroxy-1,2,3,5-tetrahydrobenzo[c]azepin-4-one, or a pharmaceuticaly acceptable salt thereof.

21. A pharmaceutical composition comprising a compound according to any preceding claim and a pharmaceutically acceptable carrier or diluent.

22. A compound as claimed in any one of claims 1 to 20, for use as a muscarinic antagonist with M₃ selectivity.

23. A compound for use as claimed in claim 22, as a bronchodilator, an antispasmodic agent, an antisecretory agent, an agent having antiulcer activity or a agent for the treatment of patients suffering from neurogenic bladder disorders.

24. A process for synthesising a compound according to claim 1, which includes the step of subjecting a compound of the formula (X): 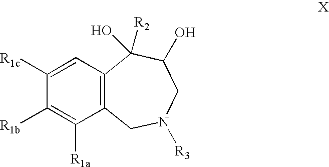
in which R1a, R1b, R1c and R2 are as defined in claim 1 and R3 is as defined in claim 1 suitably protected to oxidation conditions sufficient to oxidise the alcohol group at the 4-position of the benzo[c]azepine core to a ketone group.
